(19) United States
(12) Patent Application Publication
(10) Pub. No.: US 2026/0234596 A1
(43) Pub. Date: Aug. 13, 2026

(54) MODIFIED BILE SALT HYDROLASE ENZYMES AND METHODS OF USING THE SAME

(71) Applicant: PURPOSE BIO, INC., Beverly Hills, CA (US)

(72) Inventor: Lital GILAD-SHAOULIAN, Beverly Hills, CA (US)

(21) Appl. No.: 19/471,002

(22) PCT Filed: Apr. 1, 2024
(86) PCT No.: PCT/US2024/022506

§ 371 Date: Sep. 30, 2025

Related U.S. Application Data
(60) Provisional application No. 63/493,219, filed on Mar. 30, 2023.

(51) Int. Cl. C12N 9/80; A61K 38/00

(52) U.S. Cl. CPC C12N 9/80; C12Y 305/01024; A61K 38/00

(57) ABSTRACT

Disclosed are modified Christensenella minuta bile salt hydrolase (BSH) enzymes, prebiotics comprising the same, engineered bacterial cells comprising the modified BSH enzymes, probiotics comprising the engineered bacterial cells, methods of using the compositions to treat diseases and disorders, comprising the modified enzymes, and food products.

### CROSS-REFERENCE TO RELATED APPLICATIONS

The present application claims priority to U.S. Provisional Patent Application No. 63/493,219 that was filed Mar. 30, 2023, the entire contents of which are hereby incorporated by reference.

### SEQUENCE LISTING

A Sequence Listing accompanies this application and is submitted as an xml file of the sequence listing named “179136_00009.xml” which is 116,308 bytes in size and was created on Mar. 29, 2024. The sequence listing is electronically submitted via Patent Center and is incorporated herein by reference in its entirety.

### FIELD

The field of the disclosure relates to modified bile salt hydrolase enzymes, compositions comprising the modified enzymes, and methods of using the enzymes.

### BACKGROUND

Bile salt hydrolase (BSH) enzymes, which catalyze the deconjugation of bile acids, play a crucial role in, inter alia, the absorption of dietary fats, and regulation of the microbiome. Moreover, the function of BSH enzymes is associated with human and animal disease. Accordingly, there is a need in the art for modified BSH enzymes.

### SUMMARY

Modified Christensenella minuta Bile Salt Hydrolase Enzymes

In an aspect of the current disclosure, modified Christensenella minuta bile salt hydrolase (BSH) enzymes are provided. In some embodiments, the modified enzymes are at least 90% identical to SEQ ID NO: 1 and comprise one or more amino acid substitution at position C2, Y8, Y34, N46, H47, Y56, I57, Y64, P81, K90, F99, L127, S136, Y176, K189, S206, R207, A221, N292, or R323, relative to SEQ ID NO: 1. In some embodiments, the one or more substitutions comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K189P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1.

In an aspect of the current disclosure, modified BSH enzymes are provided. In some embodiments, the modified bile salt hydrolase (BSH) enzyme comprises amino acid substitutions at position ISV, Y8R, T9S, D11H, H12T, YT3F, R16M, L18Y, L20A, E21S, F22S, Y24E, N25G, T27K, V28I, T29V, V30I, T3TV, K33R, Y34K, F37L, H38K, F39L, R40S, K43E, L45I, N46S, H47N, M54I, Y56K, I57V, V58I, D60N, F61Y, Y64F, Y65K, D66M, T68C, S74A, G77A, L78I, N79S, D82G, N83I, D85S, K87G, V89P, K90T, E91P, Y93K, D94E, I96V, F99Y, F101L, W104Y, Q108R, A110G, S113D, R116L, I117E, E120K, Q121T, I122L, L124I, L127V, N128D, E131A, L133H, S136K, L138V, H139K, W140Y, Q145K, R146E, D147K, V151L, S153Q, F161Y, V165T, N170G, F174Y, M178L, K189N, E190K, T195K, A197S, A198P, E199S, L200I, E201K, Q204R, Y205I, R207S, A211N, L218Y, S220D, A221L, R223K, V225I, K226R, A228S, T230V, K231R, M232L, S234A, E240Y, S241D, S243Q, I244L, S245M, G252D, E255R, Q256N, Q257V, R258P, C260T, E265G, E269L, I270R, I272L, S274Q, S275T, C277I, N278D, K281R, Y284L, T287R, E290N, N292T, Y300N, E302C, N303D, D305N, N307D, T308K, S311E, Y312F, M315V, K316T, Q318P, N321Y, Y322E, R323L, N324K, and Y325K, relative to SEQ ID NO: 1. In some embodiments, the modified bile salt hydrolase (BSH) enzyme comprises amino acid substitutions at position 15V, Y8K, T9S, D11S, H12S, Y13W, R16M, L18Y, L20A, E21S, F22S, Y24E, T27E, V28I, T29V, V30I, T31V, K33R, Y34K, F37L, H38K, F39L, R40S, K43T, L45I, N46D, H47E, Y49N, M54I, Y56E, V58R, D60N, L63I, Y64F, Y65K, D66M, G77A, L78V, N79S, F80L, D82G, N83I, D85S, K87G, V89K, K90T, Y93K, D94E, F99Y, F101L, W104Y, Q108R, C109A, A110S, S113E, R116L, I117K, Q121N, I122L, L124I, L127V, N128D, L133R, S136K, L138V, H139R, W140F, S143A, Q145K, R146D, D147K, S148A, S153Q, F161Y, V165T, N170G, T173L, F174Y, M178L, K189N, E190A, T195K, A198P, E199N, L200I, E201K, Q204R, Y205I, R207A, M209D, A211N, L218K, S220D, A221L, R223K, V225I, K226R, A228T, T230V, K231R, M232L, S234A, S239T, S241D, S243Q, I244L, S245M, G252D, E255R, Q256D, Q257V, R258P, C260T, H262W, E265G, E269L, I270R, I272L, S274Q, S275V, C276A, C277I, K281T, I283K, Y284L, T287R, E290N, N292S, T295N, A296M, Y300N, E302C, N303D, D305N, G306S, N307D, T308K, Y312F, M315Q, Q318L, Q319D, N321K, Y322E, R323L, and Y325K, relative to SEQ ID NO: 1. In some embodiments, the modified bile salt hydrolase (BSH) enzyme comprises or consists of one of SEQ ID NOs: 46 or 47.

### Polynucleotides

In an aspect of the current disclosure, polynucleotides are provided. In some embodiments, the polynucleotides comprise a sequence encoding a modified Christensenella minuta bile salt hydrolase (BSH) enzyme at least 90% identical to SEQ ID NO: 1 and comprising one or more amino acid substitution at position C2, Y8, Y34, N46, H47, Y56, I57, Y64, P81, K90, F99, L127, S136, Y176, K189, S206, R207, A221, N292, or R323, relative to SEQ ID NO: 1. In some embodiments, the modified enzymes are at least 90% identical to SEQ ID NO: 1 and comprise one or more amino acid substitution at position C2, Y8, Y34, N46, H47, Y56, I57, Y64, P81, K90, F99, L127, 5136, Y176, K189, 5206, R207, A221, N292, or R323, relative to SEQ ID NO: 1. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K189P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1. In some embodiments, the polynucleotides further comprise one or more regulatory element, wherein the regulatory element is operably linked to the sequence encoding the modified enzyme. In some embodiments, the one or more regulatory element comprises a promoter, and/or enhancer. In some embodiments, the polynucleotides further comprising a selection marker. In some embodiments, the one or more regulatory element is a promoter and the promoter is a constitutive promoter. In some embodiments, the one or more regulatory element is a promoter and the promoter is an inducible promoter. In some embodiments, the promoter is inducible by environmental conditions or is responsive to low oxygen or anaerobic conditions. In some embodiments, the polynucleotide comprises one of SEQ ID NOs: 48-79.

### Prebiotics

In an aspect of the current disclosure, prebiotics are provided. In some embodiments, the prebiotics comprise a modified Christensenella minuta bile salt hydrolase (BSH) enzyme at least 90% identical to SEQ ID NO: 1 and comprising one or more amino acid substitution at position C2, Y8, Y34, N46, H47, Y56, I57, Y64, P81, K90, F99, L127, S136, Y176, K189, S206, R207, A221, N292, or R323, relative to SEQ ID NO: 1. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K1T89P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1.

### Pharmaceutical Compositions Comprising Modified BSH Enzymes

In an aspect of the current disclosure, pharmaceutical compositions are provided. In some embodiments, the pharmaceutical compositions comprise a modified Christensenella minuta bile salt hydrolase (BSH) enzyme at least 90% identical to SEQ ID NO: 1 and comprising one or more amino acid substitution at position C2, Y8, Y34, N46, H47, Y56, I57, Y64, P81, K90, F99, L127, S136, Y176, K189, S206, R207, A221, N292, or R323, relative to SEQ ID NO: 1. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K189P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1.

### Engineered Bacterial Cells

In an aspect of the current disclosure, engineered bacterial cells are provided. In some embodiments, the engineered bacterial cells comprise a polynucleotide comprising a sequence encoding a modified Christensenella minuta bile salt hydrolase (BSH) enzyme at least 90% identical to SEQ ID NO: 1 and comprising one or more amino acid substitution at position C2, Y8, Y34, N46, H47, Y56, I57, Y64, P81, K90, F99, L127, S136, Y176, K189, S206, R207, A221, N292, or R323, relative to SEQ ID NO: 1. In some embodiments, the modified enzymes are at least 90% identical to SEQ ID NO: 1 and comprise one or more amino acid substitution at position C2, Y8, Y34, N46, H47, Y56, I57, Y64, P81, K90, F99, L127, S136, Y176, K189, S206, R207, A221, N292, or R323, relative to SEQ ID NO: 1. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K189P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1. In some embodiments, the polynucleotides further comprise one or more regulatory element, wherein the regulatory element is operably linked to the sequence encoding the modified enzyme. In some embodiments, the one or more regulatory element comprises a promoter, and/or enhancer. In some embodiments, the polynucleotides further comprising a selection marker. In some embodiments, the one or more regulatory element is a promoter and the promoter is a constitutive promoter. In some embodiments, the one or more regulatory element is a promoter and the promoter is an inducible promoter. In some embodiments, the promoter is inducible by environmental conditions or is responsive to low oxygen or anaerobic conditions. In some embodiments, the polynucleotide comprises one of SEQ ID NOs: 48-79. In some embodiments, the expression of the polynucleotide is operably linked to an exogenous promoter not found in the natural Christensenella minuta genome. In some embodiments, the engineered bacterial cell is selected from the group consisting of: Acidaminococcus spp., Actinomyces spp., Akkermansia muciniphila, Allobaculum spp., Anaerococcus spp., Anaerostipes spp., Bacteroides spp., Bacteroides Other, Bacteroides acidifaciens, Bacteroides coprophilus, Bacteroides fragilis, Bacteroides ovatus, Bacteroides uniformis, Barnesiellaceae spp., Bifidobacterium adolescentis, Bifidobacterium Other, Bifidobacterium spp., Bilophila spp., Blautia obeum, Blautia producta, Blautia Other, Blautia spp., Bulleidia spp., Catenibacterium spp., Chrisenella spp., Citrobacter spp., Clostridiaceae spp., Clostridiales Other, Clostridiales spp., Clostridium perfringens, Clostridium spp., Clostridium Other, Collinsella aerofaciens, Collinsella spp., Collinsella stercoris, Coprococcus catus, Coprococcus spp., Coriobacteriaceae spp., Desulfovibrionaceae spp., Dialister spp., Dorea formicigenerans, Dorea spp., Dorea Other, Eggerthella lenta, Enterobacteriaceae Other, Enterobacteriaceae spp., Enterococcus spp., Erysipelotrichaceae spp., Eubacterium biforme, Eubacterium biforme, Eubacterium dolichum, Eubacterium spp., Faecalibacterium prausnitzii, Fusobacterium spp., Gemellaceae spp., Haemophilus parainfluenzae, Haemophilus Other, Helicobacter spp., Helicobacter Lachnospiraceae Other, Lachnospiraceae spp., Lactobacillus reuteri, Lactobacillus mucosae, Lactobacillus zeae, Lactobacillus spp., Lactobacillaceae spp., Lactococcus spp., Leuconostocaceae spp., Megamonas spp., Megasphaera spp., Methanobrevibacter spp., Mitsuokella multacida, Mitsuokella spp., Mucispirillum schaedleri, Odoribacter spp., Oscillospira spp., Parabacteroides distasonis, Parabacteroides spp., Paraprevotella spp., Paraprevotellaceae spp., Parvimonas spp., Pediococcus spp., Pediococcus Other, Peptococcus spp., Peptoniphilus spp., Peptostreptococcus anaerobius, Peptostreptococcus Other, Phascolarctobacterium spp., Prevotella copri, Prevotella spp., Prevotella stercorea, Prevotellaceae, Proteus spp., Rikenellaceae spp., Roseburia faecis, Roseburia spp., Ruminococcaceae Other, Ruminococcaceae spp., Ruminococcus bromii, Ruminococcus gnavus, Ruminococcus spp., Ruminococcus Other, Ruminococcus torques, Slackia spp., S24-7 spp., SMB53 spp., Streptococcus anginosus, Streptococcus luteciae, Streptococcus spp., Streptococcus Other, Sutterella spp., Turicibacter spp., UC Bulleidia, UC Enterobacteriaceae, UC Faecalibacterium, UC Parabacteroides, UC Pediococcus, Varibaculum spp., Veillonella spp., Sutterella, Turicibacter, UC Clostridiales, UC Erysipelotrichaceae, UC Ruminococcaceae, Veillonella parvula, Veillonella spp., Veillonella dispar, and Weissella. In some embodiments, the polynucleotide is integrated into a genome of the bacterial cell.

### Probiotics

In an aspect of the current disclosure, probiotics are provided. In some embodiments, the probiotics comprise a engineered bacterial cell comprising a polynucleotide comprising a sequence encoding a modified Christensenella minuta bile salt hydrolase (BSH) enzyme at least 90% identical to SEQ ID NO: 1 and comprising one or more amino acid substitution at position A140, N274, C2, F68, L137, H212, 159, S103, K92, K312, L20, V22, L67, P225, or Y26, relative to SEQ ID NO: 1. In some embodiments, the modified enzymes are at least 90% identical to SEQ ID NO: 1 and comprise one or more amino acid substitution at position C2, Y8, Y34, N46, H47, Y56, I57, Y64, P81, K90, F99, L127, S136, Y176, K189, S206, R207, A221, N292, or R323, relative to SEQ ID NO: 1. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises The modified enzyme of claim 1, wherein the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K189P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1. In some embodiments, the polynucleotides further comprise one or more regulatory element, wherein the regulatory element is operably linked to the sequence encoding the modified enzyme. In some embodiments, the one or more regulatory element comprises a promoter, and/or enhancer. In some embodiments, the polynucleotides further comprising a selection marker. In some embodiments, the one or more regulatory element is a promoter and the promoter is a constitutive promoter. In some embodiments, the one or more regulatory element is a promoter and the promoter is an inducible promoter. In some embodiments, the promoter is inducible by environmental conditions or is responsive to low oxygen or anaerobic conditions. In some embodiments, the polynucleotide comprises one of SEQ ID NOs: 48-79. In some embodiments, the expression of the polynucleotide is operably linked to an exogenous promoter not found in the natural Christensenella minuta genome. In some embodiments, the engineered bacterial cell is selected from the group consisting of: Acidaminococcus spp., Actinomyces spp., Akkermansia muciniphila, Allobaculum spp., Anaerococcus spp., Anaerostipes spp., Bacteroides spp., Bacteroides Other, Bacteroides acidifaciens, Bacteroides coprophilus, Bacteroides fragilis, Bacteroides ovatus, Bacteroides uniformis, Barnesiellaceae spp., Bifidobacterium adolescentis, Bifidobacterium Other, Bifidobacterium spp., Bilophila spp., Blautia obeum, Blautia producta, Blautia Other, Blautia spp., Bulleidia spp., Catenibacterium spp., Chrisenella spp., Citrobacter spp., Clostridiaceae spp., Clostridiales Other, Clostridiales spp., Clostridium perfringens, Clostridium spp., Clostridium Other, Collinsella aerofaciens, Collinsella spp., Collinsella stercoris, Coprococcus catus, Coprococcus spp., Coriobacteriaceae spp., Desulfovibrionaceae spp., Dialister spp., Dorea formicigenerans, Dorea spp., Dorea Other, Eggerthella lenta, Enterobacteriaceae Other, Enterobacteriaceae spp., Enterococcus spp., Erysipelotrichaceae spp., Eubacterium biforme, Eubacterium biforme, Eubacterium dolichum, Eubacterium spp., Faecalibacterium prausnitzii, Fusobacterium spp., Gemellaceae spp., Haemophilus parainfluenzae, Haemophilus Other, Helicobacter spp., Helicobacter Lachnospiraceae Other, Lachnospiraceae spp., Lactobacillus reuteri, Lactobacillus mucosae, Lactobacillus zeae, Lactobacillus spp., Lactobacillaceae spp., Lactococcus spp., Leuconostocaceae spp., Megamonas spp., Megasphaera spp., Methanobrevibacter spp., Mitsuokella multacida, Mitsuokella spp., Mucispirillum schaedleri, Odoribacter spp., Oscillospira spp., Parabacteroides distasonis, Parabacteroides spp., Paraprevotella spp., Paraprevotellaceae spp., Parvimonas spp., Pediococcus spp., Pediococcus Other, Peptococcus spp., Peptoniphilus spp., Peptostreptococcus anaerobius, Peptostreptococcus Other, Phascolarctobacterium spp., Prevotella copri, Prevotella spp., Prevotella stercorea, Prevotellaceae, Proteus spp., Rikenellaceae spp., Roseburia faecis, Roseburia spp., Ruminococcaceae Other, Ruminococcaceae spp., Ruminococcus bromii, Ruminococcus gnavus, Ruminococcus spp., Ruminococcus Other, Ruminococcus torques, Slackia spp., S24-7 spp., SMB53 spp., Streptococcus anginosus, Streptococcus luteciae, Streptococcus spp., Streptococcus Other, Sutterella spp., Turicibacter spp., UC Bulleidia, UC Enterobacteriaceae, UC Faecalibacterium, UC Parabacteroides, UC Pediococcus, Varibaculum spp., Veillonella spp., Sutterella, Turicibacter, UC Clostridiales, UC Erysipelotrichaceae, UC Ruminococcaceae, Veillonella parvula, Veillonella spp., Veillonella dispar, and Weissella. In some embodiments, the polynucleotide is integrated into a genome of the bacterial cell.

### Pharmaceutical Compositions Comprising Engineered Bacterial Cells

In an aspect of the current disclosure, further pharmaceutical compositions are provided. In some embodiments, the pharmaceutical compositions comprise and engineered bacterial cell comprising a polynucleotide comprising a sequence encoding a modified Christensenella minuta bile salt hydrolase (BSH) enzyme at least 90% identical to SEQ ID NO: 1 and comprising one or more amino acid substitution at position C2, Y8, Y34, N46, H47, Y56, I57, Y64, P81, K90, F99, L127, S136, Y176, K189, 5206, R207, A221, N292, or R323, relative to SEQ ID NO: 1. In some embodiments, the modified enzymes are at least 90% identical to SEQ ID NO: 1 and comprise one or more amino acid substitution at position C2, Y8, Y34, N46, H47, Y56, I57, Y64, P81, K90, F99, L127, 5136, Y176, K189, 5206, R207, A221, N292, or R323, relative to SEQ ID NO: 1. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises The modified enzyme of claim 1, wherein the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K189P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1. In some embodiments, the polynucleotides further comprise one or more regulatory element, wherein the regulatory element is operably linked to the sequence encoding the modified enzyme. In some embodiments, the one or more regulatory element comprises a promoter, and/or enhancer. In some embodiments, the polynucleotides further comprising a selection marker. In some embodiments, the one or more regulatory element is a promoter and the promoter is a constitutive promoter. In some embodiments, the one or more regulatory element is a promoter and the promoter is an inducible promoter. In some embodiments, the promoter is inducible by environmental conditions or is responsive to low oxygen or anaerobic conditions. In some embodiments, the polynucleotide comprises one of SEQ ID NOs: 48-79. In some embodiments, the expression of the polynucleotide is operably linked to an exogenous promoter not found in the natural Christensenella minuta genome. In some embodiments, the engineered bacterial cell is selected from the group consisting of: Acidaminococcus spp., Actinomyces spp., Akkermansia muciniphila, Allobaculum spp., Anaerococcus spp., Anaerostipes spp., Bacteroides spp., Bacteroides Other, Bacteroides acidifaciens, Bacteroides coprophilus, Bacteroides fragilis, Bacteroides ovatus, Bacteroides uniformis, Barnesiellaceae spp., Bifidobacterium adolescentis, Bifidobacterium Other, Bifidobacterium spp., Bilophila spp., Blautia obeum, Blautia producta, Blautia Other, Blautia spp., Bulleidia spp., Catenibacterium spp., Chrisenella spp., Citrobacter spp., Clostridiaceae spp., Clostridiales Other, Clostridiales spp., Clostridium perfringens, Clostridium spp., Clostridium Other, Collinsella aerofaciens, Collinsella spp., Collinsella stercoris, Coprococcus catus, Coprococcus spp., Coriobacteriaceae spp., Desulfovibrionaceae spp., Dialister spp., Dorea formicigenerans, Dorea spp., Dorea Other, Eggerthella lenta, Enterobacteriaceae Other, Enterobacteriaceae spp., Enterococcus spp., Erysipelotrichaceae spp., Eubacterium biforme, Eubacterium biforme, Eubacterium dolichum, Eubacterium spp., Faecalibacterium prausnitzii, Fusobacterium spp., Gemellaceae spp., Haemophilus parainfluenzae, Haemophilus Other, Helicobacter spp., Helicobacter Lachnospiraceae Other, Lachnospiraceae spp., Lactobacillus reuteri, Lactobacillus mucosae, Lactobacillus zeae, Lactobacillus spp., Lactobacillaceae spp., Lactococcus spp., Leuconostocaceae spp., Megamonas spp., Megasphaera spp., Methanobrevibacter spp., Mitsuokella multacida, Mitsuokella spp., Mucispirillum schaedleri, Odoribacter spp., Oscillospira spp., Parabacteroides distasonis, Parabacteroides spp., Paraprevotella spp., Paraprevotellaceae spp., Parvimonas spp., Pediococcus spp., Pediococcus Other, Peptococcus spp., Peptoniphilus spp., Peptostreptococcus anaerobius, Peptostreptococcus Other, Phascolarctobacterium spp., Prevotella copri, Prevotella spp., Prevotella stercorea, Prevotellaceae, Proteus spp., Rikenellaceae spp., Roseburia faecis, Roseburia spp., Ruminococcaceae Other, Ruminococcaceae spp., Ruminococcus bromii, Ruminococcus gnavus, Ruminococcus spp., Ruminococcus Other, Ruminococcus torques, Slackia spp., S24-7 spp., SMB53 spp., Streptococcus anginosus, Streptococcus luteciae, Streptococcus spp., Streptococcus Other, Sutterella spp., Turicibacter spp., UC Bulleidia, UC Enterobacteriaceae, UC Faecalibacterium, UC Parabacteroides, UC Pediococcus, Varibaculum spp., Veillonella spp., Sutterella, Turicibacter, UC Clostridiales, UC Erysipelotrichaceae, UCRuminococcaceae, Veillonella parvula, Veillonella spp., Veillonella dispar, and Weissella. In some embodiments, the polynucleotide is integrated into a genome of the bacterial cell.

### Methods

In an aspect of the current disclosure, methods are provided. In some embodiments, the methods comprise administering a pharmaceutical composition comprising a modified BSH enzyme at least 90% identical to SEQ ID NO: 1 and comprise one or more amino acid substitution at position C2, Y8, Y34, N46, H47, Y56, I57, Y64, P81, K90, F99, L127, 5136, Y176, K189, 5206, R207, A221, N292, or R323, relative to SEQ ID NO: 1 to a subject in need thereof. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises The modified enzyme of claim 1, wherein the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K1T89P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1. In some embodiments, the subject in need thereof is suffering from one or more of: non-alcoholic fatty liver disease (NAFLD), non-alcoholic fatty liver disease (NAFLD), a disease or disorder associated with metabolic syndrome, or cardiovascular disease.

In some embodiments, methods of treating non-alcoholic fatty liver disease (NAFLD) in a subject in need thereof are provided and comprise administering a therapeutically effective amount of a pharmaceutical composition comprising a modified enzyme at least 90% identical to SEQ ID NO: 1 and comprising one or more amino acid substitution at position C2, Y8, Y34, N46, H47, Y56, I57, Y64, P81, K90, F99, L127, S136, Y176, K189, S206, R207, A221, N292, or R323, relative to SEQ ID NO: 1 to the subject to treat the NAFLD. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises The modified enzyme of claim 1, wherein the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K1T89P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1.

In some embodiments, method of treating non-alcoholic steatohepatitis (NASH) in a subject in need thereof are provided and the methods comprise administering a therapeutically effective amount of a pharmaceutical composition comprising a modified enzyme at least 90% identical to SEQ ID NO: 1 and comprising one or more amino acid substitution at position C2, Y8, Y34, N46, H47, Y56, I57, Y64, P81, K90, F99, L127, S136, Y176, K189, S206, R207, A221, N292, or R323, relative to SEQ ID NO: 1 to the subject to treat the NASH. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises The modified enzyme of claim 1, wherein the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K189P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1.

In some embodiments, method of treating liver cancer in a subject in need thereof are provided and comprise administering a therapeutically effective amount of a pharmaceutical composition comprising a modified enzyme at least 90% identical to SEQ ID NO: 1 and comprising one or more amino acid substitution at position C2, Y8, Y34, N46, H47, Y56, I57, Y64, P81, K90, F99, L127, S136, Y176, K189, S206, R207, A221, N292, or R323, relative to SEQ ID NO: 1 the subject to treat the liver cancer. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises The modified enzyme of claim 1, wherein the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K189P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1.

In some embodiments, methods of treating Alzheimer's disease in a subject in need thereof are provided and comprise administering a therapeutically effective amount of a pharmaceutical composition comprising modified enzymes are at least 90% identical to SEQ ID NO: 1 and comprise one or more amino acid substitution at position C2, Y8, Y34, N46, H47, Y56, 157, Y64, P81, K90, F99, L127, 5136, Y176, K189, 5206, R207, A221, N292, or R323, relative to SEQ ID NO: 1 to the subject to treat Alzheimer's disease. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises The modified enzyme of claim 1, wherein the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K1T89P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1.

In some embodiments, methods of treating a disease or disorder associated with metabolic syndrome in a subject in need thereof are provided and comprise administering a therapeutically effective amount of a pharmaceutical composition comprising a modified enzyme are at least 90% identical to SEQ ID NO: 1 and comprise one or more amino acid substitution at position C2, Y8, Y34, N46, H47, Y56, I57, Y64, P81, K90, F99, L127, 5136, Y176, K189, 5206, R207, A221, N292, or R323, relative to SEQ ID NO: 1 to the subject to treat the disease or disorder associated with metabolic syndrome. In some embodiments, the one or more substitution comprises Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q. In some embodiments, the one or more substitution comprises Y8V, Y34K, I57V, Y64S, P81A, K90E, L127I, S136A, K189P, K189N, S206G, R207Q, N292S, R323L, or R323E. In some embodiments, the one or more substitution comprises I57V. In some embodiments, the one or more substitution comprises The modified enzyme of claim 1, wherein the one or more substitution comprises Y64S. In some embodiments, the one or more substitution comprises S136A. In some embodiments, the one or more substitution comprises K189P. In some embodiments, the one or more substitution comprises Y8V, P81A, S206G, and R207Q. In some embodiments, the one or more substitution comprises Y34K, I57V, L127I, and N292S. In some embodiments, the one or more substitution comprises Y56T, I57V, L127I, and N292S. In some embodiments, the one or more substitution comprises K90E, L127I, N292S, and R323E. In some embodiments, the one or more substitution comprises Y34N, N46K, H47N, Y64F, Y176K, and A221L. In some embodiments, the one or more substitution comprises Y8T, Y34N, Y56V, I57V, F99Y, L127I, S136A, K189N, N292S, and R323L. In some embodiments, the one or more substitution comprises C2S. In some embodiments, the modified enzyme comprises or consists of one of SEQ ID NOs: 2-47. In some embodiments, the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5. In some embodiments, the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6. In some embodiments, the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8. In some embodiments, the modified enzyme comprises a sequence with at least 90% identity to SEQ ID NO: 9 and wherein the modified enzyme has the substitution K189P, with regard to SEQ ID NO: 1, optionally, wherein the modified enzyme comprises or consists of SEQ ID NO: 9. In some embodiments, the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36. In some embodiments, the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37. In some embodiments, the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41. In some embodiments, the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42. In some embodiments, the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43. In some embodiments, the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44. In some embodiments, the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45. In some embodiments, the modified enzyme has increased activity compared to wild type C. minuta BSH at about pH 5. In some embodiments, the modified enzyme has increased activity compared to wild type C. minuta BSH at about pH 7. In some embodiments, the modified Christensenella minuta bile salt hydrolase (BSH) enzyme has at least 90% identity to SEQ ID NO: 1, wherein the modified BSH enzyme has at least one substitution modification relative to SEQ ID NO: 1. In some embodiments, the disease or disorder associated with metabolic syndrome is selected from the group consisting of: hyperlipidemia, hypercholesteremia, obesity, and cardiovascular disease. In some embodiments, the cardiovascular disease is selected from the group consisting of coronary artery disease, peripheral artery disease, carotid artery disease, heart failure, and stroke.

In an aspect of the current disclosure, medical foods are provided. In some embodiments, the medical foods comprise the modified BSH enzymes of the instant disclosure, the prebiotics of the instant disclosure, the probiotics of the instant disclosure, or the bacterial cells of the instant disclosure.

In an aspect of the current disclosure, methods of making a modified bile salt hydrolase (BSH) enzyme are provided and comprise introducing a polynucleotide of the instant disclosure into a cell, thereby causing the cell to express the modified BSH enzyme. In some embodiments, the methods further comprise enriching, purifying, or isolating the modified BSH enzyme. In some embodiments, the cell is a mammalian cell, an insect cell, a fungal cell, or a bacterial cell.

### BRIEF DESCRIPTION OF THE FIGURES

FIG. 1 shows an exemplary enzymatic reaction with the disclosed BSH enzymes.

FIG. 2 shows an exemplary enzymatic reaction with the disclosed BSH enzymes.

FIGS. 3A and 3B show exemplary data demonstrating GCA hydrolysis by the disclosed enzymes and glycine detection.

FIGS. 4A and 4B show exemplary data demonstrating GCDCA hydrolysis by the disclosed enzymes and glycine detection.

FIGS. 5A, 5B, 5C, 5D, 5E, and 5F show an exemplary polyacrylamide gel demonstrating successful expression of the indicated modified BSH enzymes. Staining: Fixation for 60 min in staining solution (25% (v/v) isopropanol, 10% (v/v) acetic acid, 65% (v/v) H2O) afterward staining for 60 min in staining solution (10% acetic acid (v/v), 0.006% (w/v) Coomassie brilliant blue R-250, 90% H2O). 1_BSH_WT is an unrelated BSH enzyme control that is distinct from the C. minuta BSH of the instant disclosure. BSH2_WT is WT C. minuta BSH, i.e., SEQ ID NO: 1.

FIGS. 6A and 6B show pie charts describing the equilibrium of BA in healthy, NASH and NAFLD states. The data for the charts is derived from Puri, P. et al., THE PRESENCE AND SEVERITY OF NONALCOHOLIC STEATOHEPATITIS IS ASSOCIATED WITH SPECIFIC CHANGES IN CIRCULATING BILE ACIDS. Hepatology. 2018 February; 67(2): 534-548, which is incorporated by reference herein in its entirety.

FIG. 7 shows pie charts demonstrating the novel approach disclosed herein to shifting the BA equilibrium back to healthy from disease states, leveraging the disclosed novel enzymes, which have increased BA deconjugating activity.

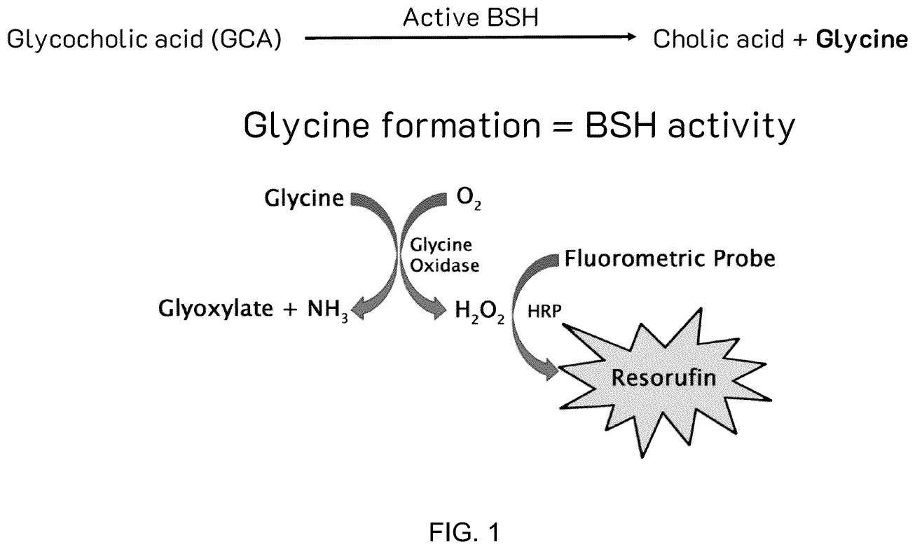

### DETAILED DESCRIPTION

This disclosure provides modified Christensenella minuta bile salt hydrolase (BSH) enzymes, pharmaceutical compositions comprising the modified BSH enzymes, polynucleotides encoding the modified BSH enzymes, cells, including but not limited to bacterial cells, comprising the modified BSH enzymes, cells, including but not limited to bacterial cells comprising the disclosed polynucleotides, and methods of using the modified BSH enzymes, cells (e.g., bacterial cells), and pharmaceutical compositions as probiotics or therapeutics for the treatment of human and animal disease, the improvement of livestock and companion animal health and growth, agricultural, and biofuel, applications. The disclosed compositions and methods are intended to shift the bile acid equilibrium to a healthy state, while keeping the BA pool intact.

Bile salt hydrolases catalyze the hydrolysis of “conjugated” bile acids to “unconjugated” bile acids, liberating an amino acid, e.g., glycine or taurine. For example, the disclosed modified BSH enzymes have been experimentally shown to hydrolyze the bile acid Glycochenodeoxycholic acid (GCDCA) to result in chenodeoxycholic acid and free glycine and hydrolyze glycocholic acid to result in cholic acid and glycine. However, the disclosed modified BSH enzymes are believed to hydrolyze additional bile acid species.

As used herein, “engineered BSH” or “modified BSH” are used interchangeably. Further, in the context of this disclosure, BSH is understood to refer to Christensenella minuta BSH, unless otherwise specified.

Modified Christensenella minuta Bile Salt Hydrolase Enzymes

In an aspect of the current disclosure, modified Christensenella minuta bile salt hydrolase enzymes are provided. The modified enzymes comprise one or more amino acid substitution at position C2, Y8, Y34, N46, H47, Y56, I57, Y64, P81, K90, F99, L127, 5136, Y176, K189, 5206, R207, A221, N292, or R323, with regard to SEQ ID NO: 1.

SEQ ID NO: 1 is:
MCTAITYYTKDHYFGRNLDLEFSYNETVTVTPKYYPFHFRNGKVLNHHYA
MIGMAYIVDDFPLYYDATNEKGLSMAGLNFPDNADYKEVKEGYDNIAPFE
FIPWILGQCASVSEARILLEQINLVNLNFSEELPLSPLHWMISDQRDSIV
VESTKDGLKVFENPVGVLTNNPTFDYQMFNLNNYMHLSKEPPANTFAAEL
ELEQYSRGMGAIGLPGDLSSASRFVKAAFTKMNSVSGDSESESISQFFHI
LGSVEQQRGCVHLGEDKYEITIYSSCCNMDKGIYYYTTYENNQITAVDMY
KENLDGNTIISYPLMKEQQINYRNY

As used herein, “substitution” or “substitution modification” are used interchangeably and refer to the replacement of one amino acid with another amino acid. The disclosed modified enzymes may be at least about 90% identical to SEQ ID NO: 1. The disclosed modified BSH enzymes may be at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to SEQ ID NO: 1, which is the amino acid sequence of a wild type C. minuta bile salt hydrolase. The one or more substitution may comprise C2S, Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q.

The one or more substitution may comprise Y8V, Y34K, I57V, Y64S, P81A, K90E, L127I, S136A, K189P, K189N, S206G, R207Q, N292S, R323L, or R323E. The one or more substitution may comprise I57V. The one or more substitution may comprise Y64S. The one or more substitution may comprise S136A. The one or more substitution may comprise K189P. The one or more substitution may comprise Y8V, P81A, S206G, and R207Q. The one or more substitution may comprise Y34K, I57V, L127I, and N292S. The one or more substitution may comprise Y56T, I57V, L127I, and N292S. The one or more substitution may comprise K90E, L127I, N292S, and R323E. The one or more substitution may comprise Y34N, N46K, H47N, Y64F, Y176K, and A221L. The one or more substitution may comprise Y8T, Y34N, Y56V, I57V, F99Y, L127I, S136A, K189N, N292S, and R323L. The one or more substitution may comprise C2S. The modified enzyme may comprise one of SEQ ID NOs: 2-47. The modified enzyme may comprise one of SEQ ID NOs: 5, 6, 8, 9, 36, 37, and 41-47.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 5 and wherein the modified enzyme has the substitution I57V, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 6 and wherein the modified enzyme has the substitutions Y64S, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 8 and wherein the modified enzyme has the substitution S136A, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 9 and wherein the modified enzyme has the substitutions K189P, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 36 and wherein the modified enzyme has the substitutions Y8V, P81A, S206G, and R207Q, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 37 and wherein the modified enzyme has the substitutions Y34K, I57V, L127I, and N292S, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 41 and wherein the modified enzyme has the substitutions Y56T, I57V, L127I, and N292S, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 42 and wherein the modified enzyme has the substitutions K90E, L127I, N292S, and R323E, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 43 and wherein the modified enzyme has the substitutions Y34N, H47N, Y64F, Y176K, A221P, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 44 and wherein the modified enzyme has the substitutions Y34N, N46K, H47N, Y64F, Y176K, and A221L, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1.

The modified enzyme may comprise a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to any one of SEQ ID NOs: 2-47 and wherein the modified enzyme has the substitutions indicated in Table 1, with regard to SEQ ID NO: 1.

The modified enzyme may comprise increased activity compared to wild type C. minuta BSH at about pH 5. The modified enzyme may comprise increased activity compared to wild type C. minuta BSH at about pH 7. The modified enzyme may comprise increased activity compared to wild type C. minuta BSH at about pH 1, 2, 3, 4, 5, 6, 7, 8, 9, 10, 11, 12, 13, or 14.

In an aspect of the instant disclosure modified BSH enzymes are provided. The modified BSH enzymes may comprise amino acid substitutions at position ISV, Y8R, T9S, D11H, H12T, Y13F, R16M, L18Y, L20A, E21S, F22S, Y24E, N25G, T27K, V28I, T29V, V30I, T31V, K33R, Y34K, F37L, H38K, F39L, R40S, K43E, L45I, N46S, H47N, M54I, Y56K, I57V, V58I, D60N, F61Y, Y64F, Y65K, D66M, T68C, S74A, G77A, L78I, N79S, D82G, N83I, D85S, K87G, V89P, K90T, E91P, Y93K, D94E, I96V, F99Y, F101L, W104Y, Q108R, A110G, S113D, R116L, I117E, E120K, Q121T, I122L, L124I, L127V, N128D, E131A, L133H, S136K, L138V, H139K, W140Y, Q145K, R146E, D147K, V151L, S153Q, F161Y, V165T, N170G, F174Y, M178L, K189N, E190K, T195K, A197S, A198P, E199S, L200I, E201K, Q204R, Y205I, R207S, A211N, L218Y, S220D, A221L, R223K, V225I, K226R, A228S, T230V, K231R, M232L, S234A, E240Y, S241D, S243Q, I244L, S245M, G252D, E255R, Q256N, Q257V, R258P, C260T, E265G, E269L, I270R, I272L, S274Q, S275T, C277I, N278D, K281R, Y284L, T287R, E290N, N292T, Y300N, E302C, N303D, D305N, N307D, T308K, S311E, Y312F, M315V, K316T, Q318P, N321Y, Y322E, R323L, N324K, and Y325K, relative to SEQ ID NO: 1. The modified enzymes may comprise SEQ ID NO: 46, or a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 46.

The modified BSH enzymes may comprise amino acid substitutions at position I5V, Y8K, T9S, D11S, H12S, Y13W, R16M, L18Y, L20A, E21S, F22S, Y24E, T27E, V28I, T29V, V30I, T31V, K33R, Y34K, F37L, H38K, F39L, R40S, K43T, L45I, N46D, H47E, Y49N, M54I, Y56E, V58R, D60N, L63I, Y64F, Y65K, D66M, G77A, L78V, N79S, F80L, D82G, N83I, D85S, K87G, V89K, K90T, Y93K, D94E, F99Y, F101L, W104Y, Q108R, C109A, A110S, S113E, R116L, I117K, Q121N, I122L, L124I, L127V, N128D, L133R, S136K, L138V, H139R, W140F, S143A, Q145K, R146D, D147K, S148A, S153Q, F161Y, V165T, N170G, T173L, F174Y, M178L, K189N, E190A, T195K, A198P, E199N, L200I, E201K, Q204R, Y205I, R207A, M209D, A211N, L218K, S220D, A221L, R223K, V225I, K226R, A228T, T230V, K231R, M232L, S234A, S239T, S241D, S243Q, I244L, S245M, G252D, E255R, Q256D, Q257V, R258P, C260T, H262W, E265G, E269L, I270R, I272L, S274Q, S275V, C276A, C277I, K281T, I283K, Y284L, T287R, E290N, N292S, T295N, A296M, Y300N, E302C, N303D, D305N, G306S, N307D, T308K, Y312F, M315Q, Q318L, Q319D, N321K, Y322E, R323L, and Y325K, relative to SEQ ID NO: 1. The modified enzymes may comprise SEQ ID NO: 47, or a sequence with at least at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to SEQ ID NO: 47.

The modified enzyme may comprise any one of SEQ ID NOs: 2-47, or a sequence with at least at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to any one of SEQ ID NOs: 2-47. The modified enzyme may comprise any one of disclosed substitution mutations disclosed herein, e.g., see Table 1.

Several C. minuta BSH mutants have been generated and are listed below to correspond the experimental naming convention to the identity of the mutant, e.g., BSH2_3 which comprises the I57V mutation relative to SEQ ID NO: 1.

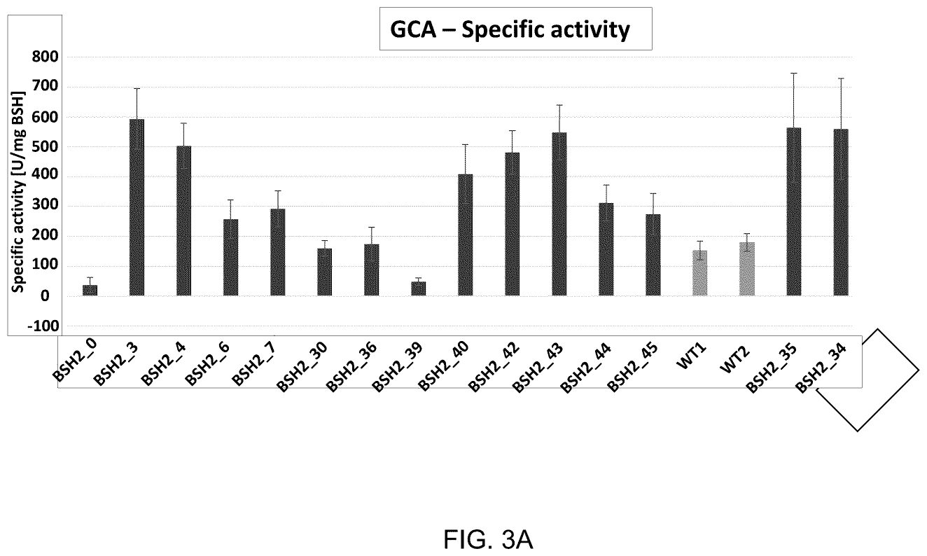

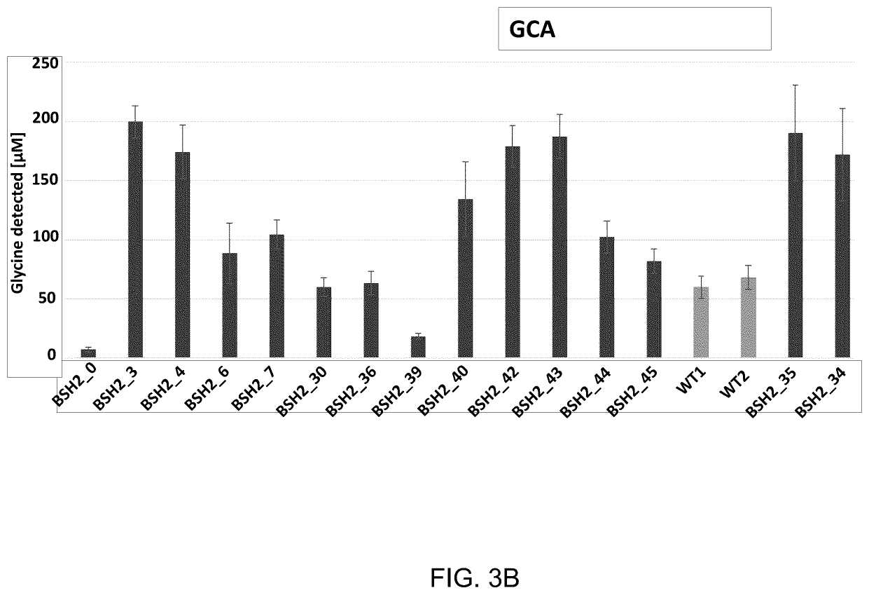

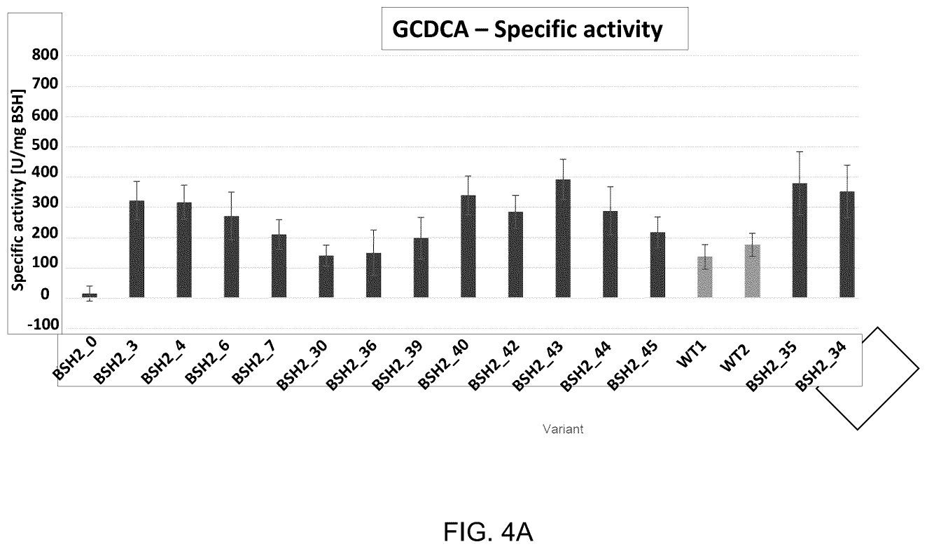

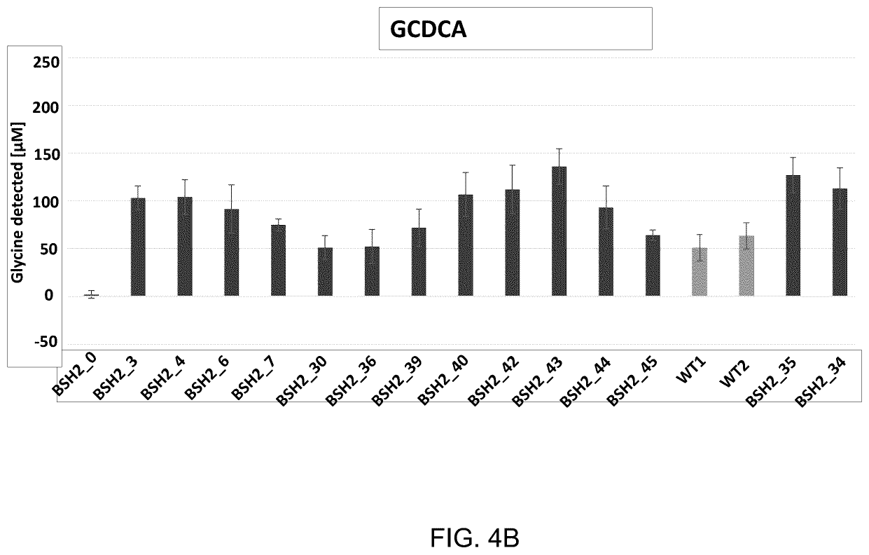

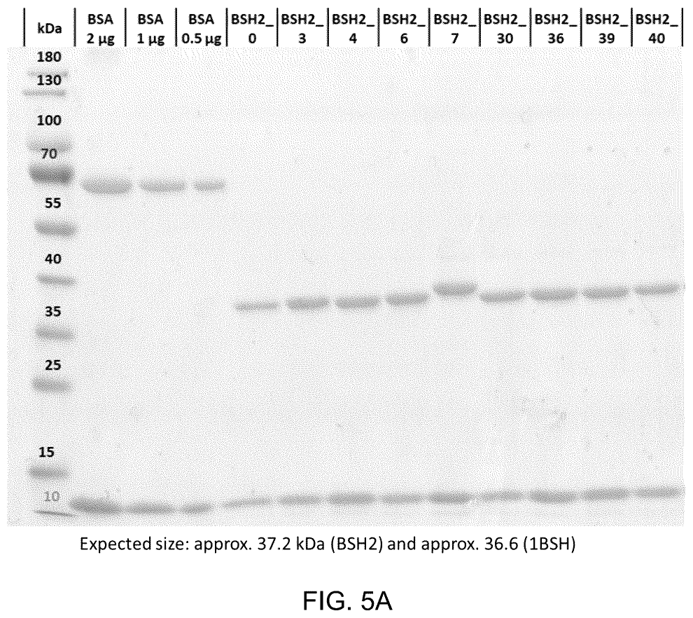

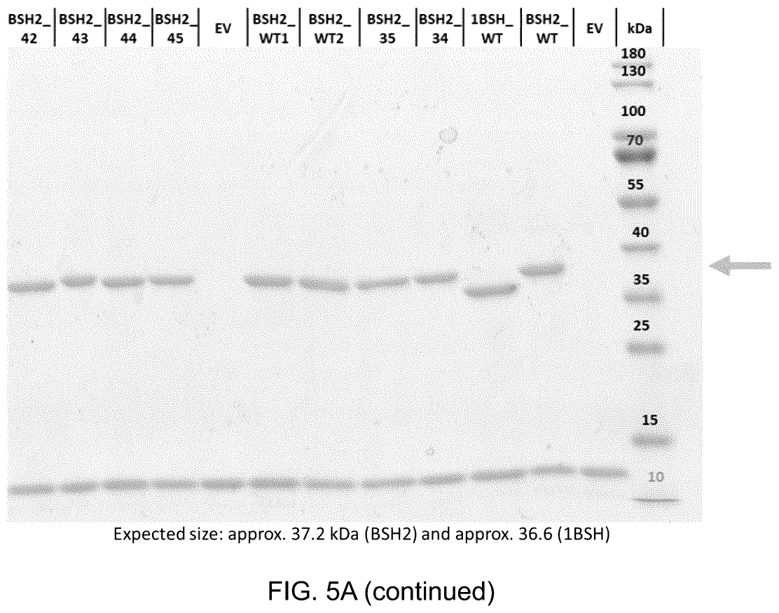

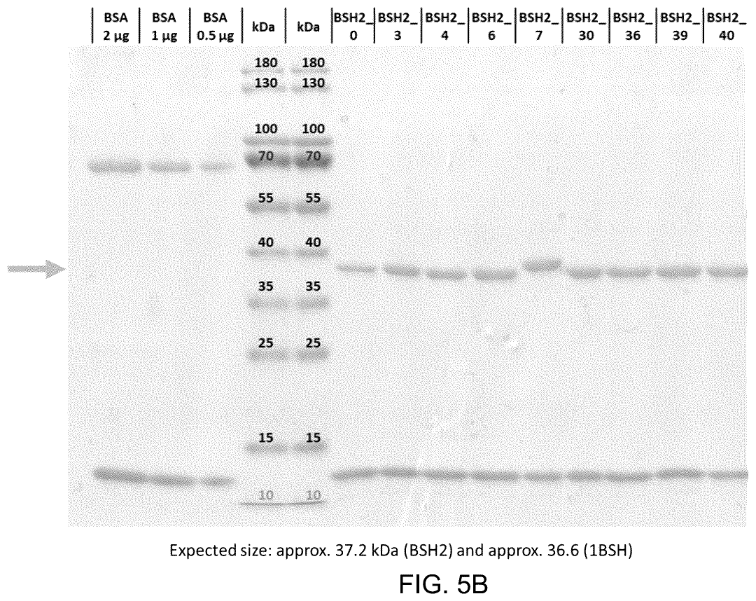

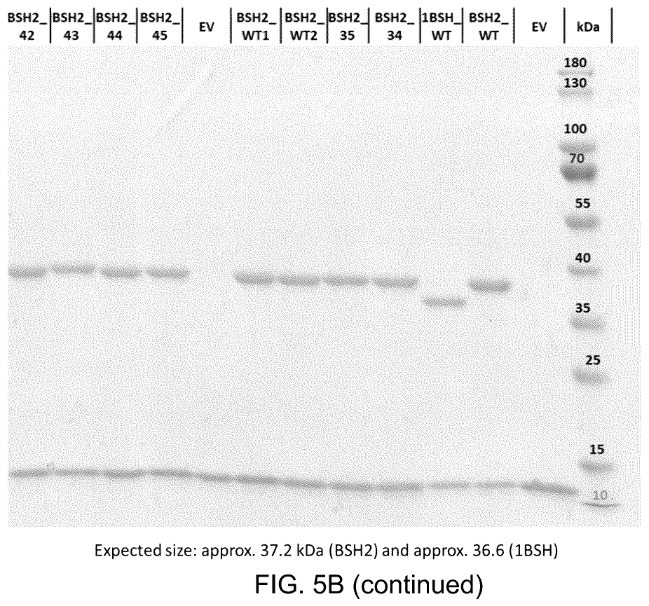

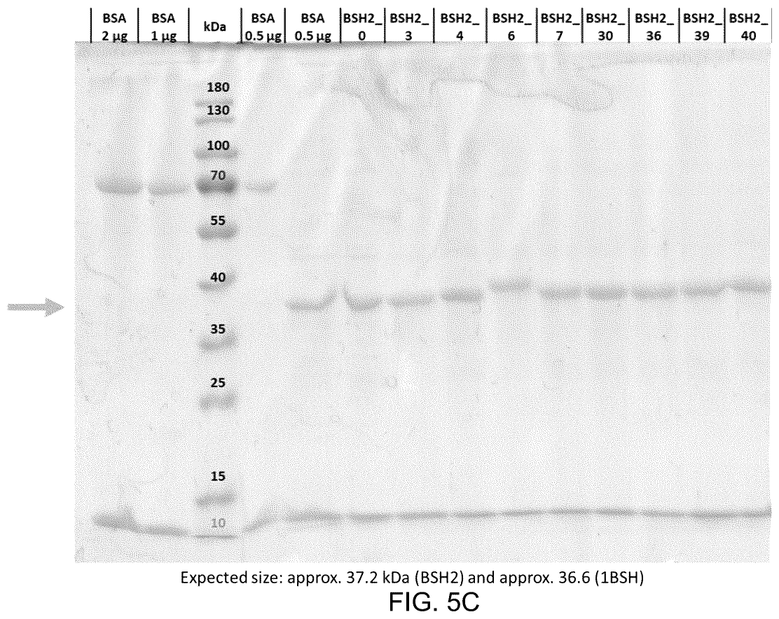

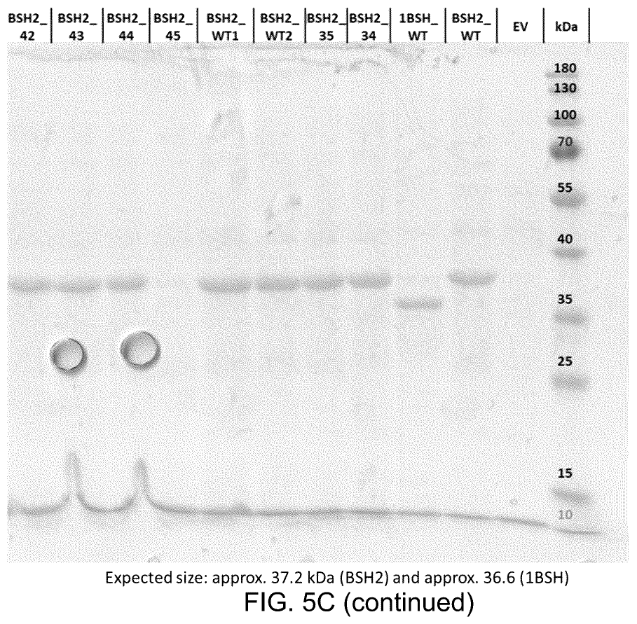

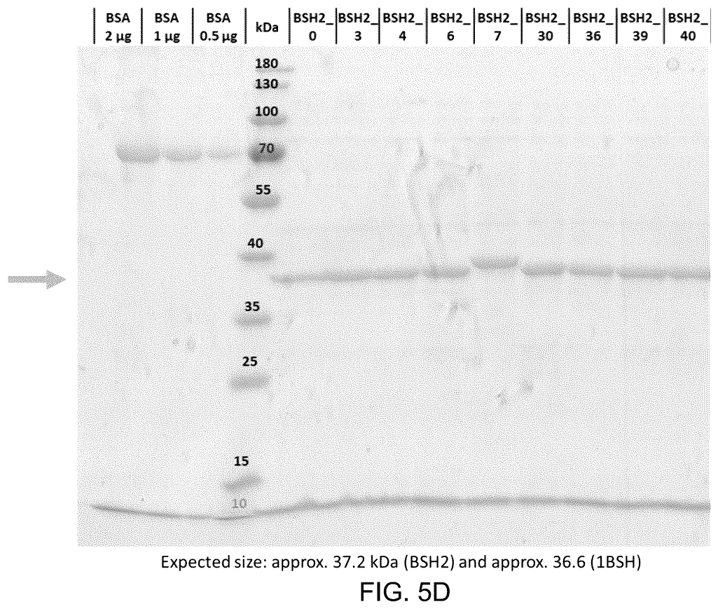

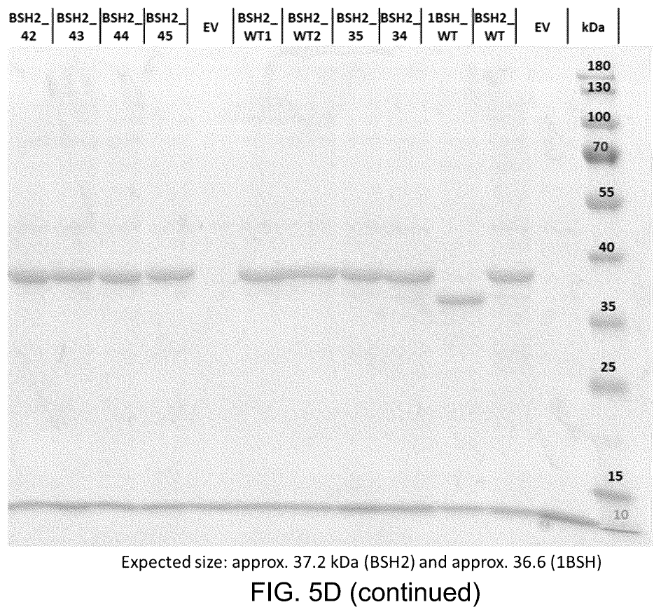

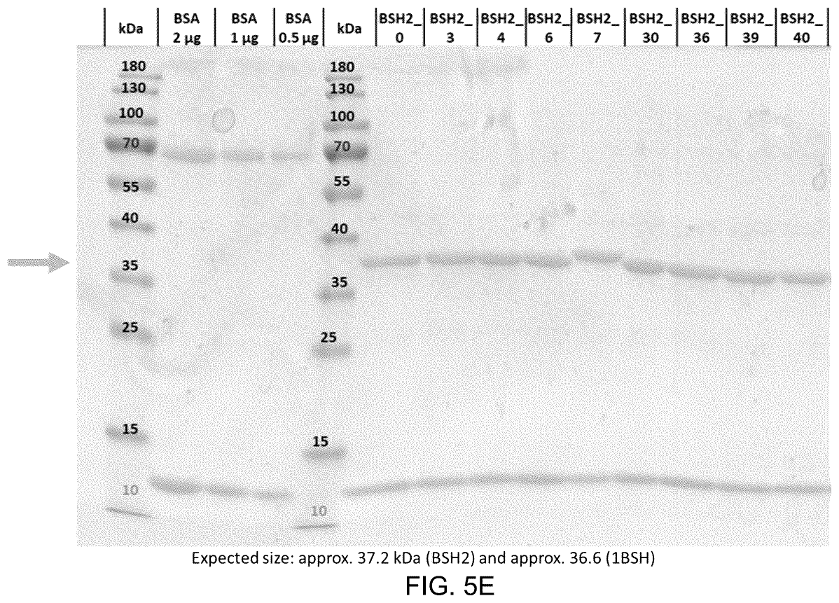

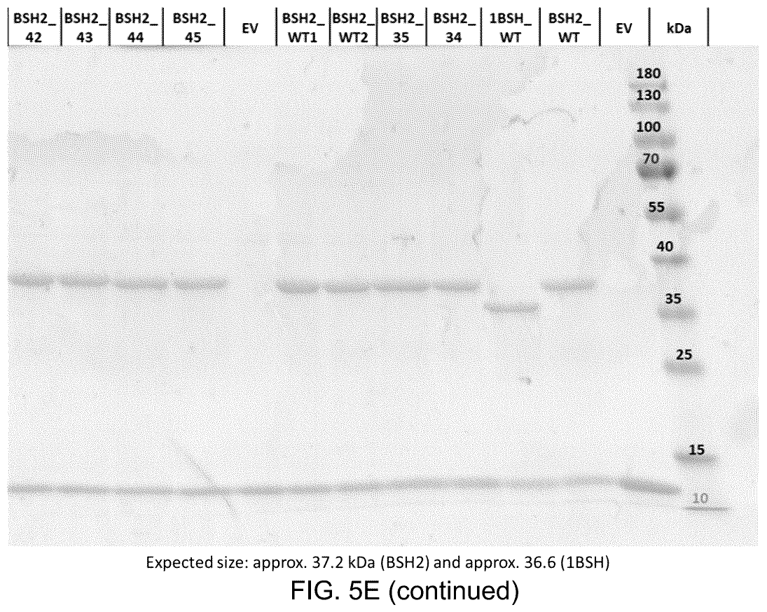

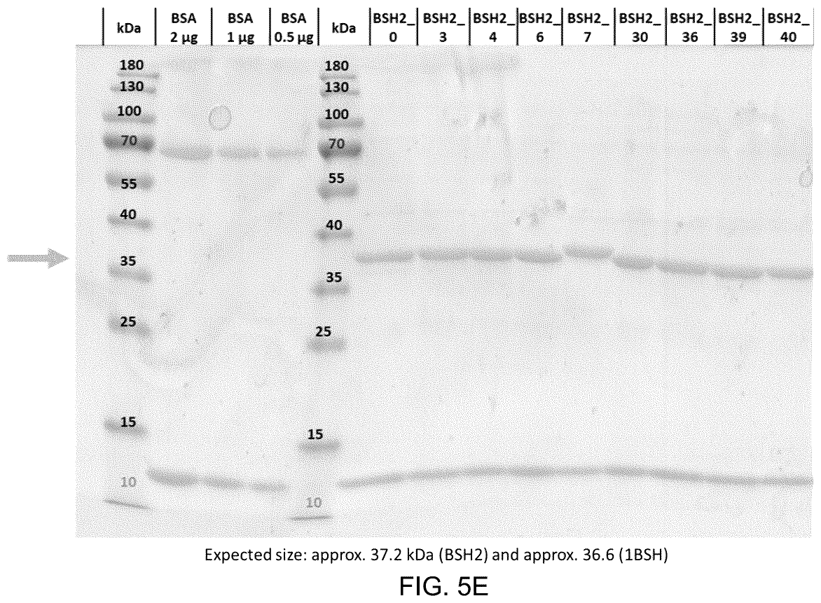

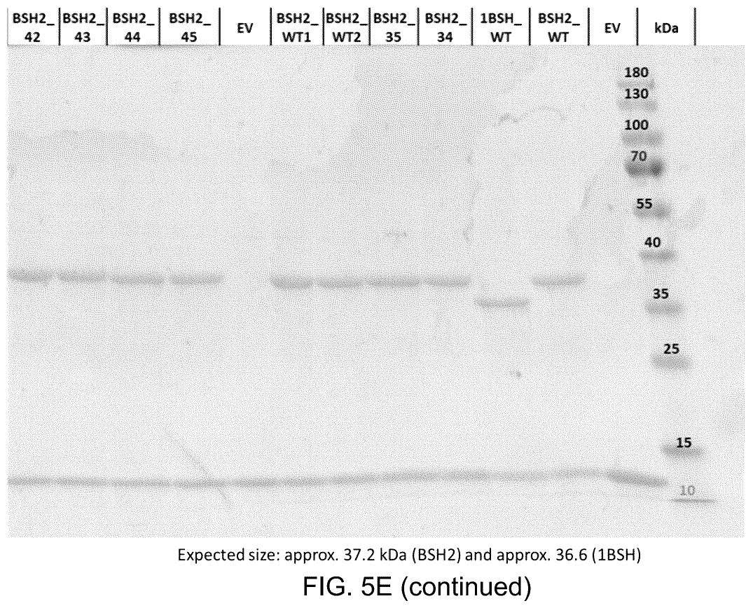

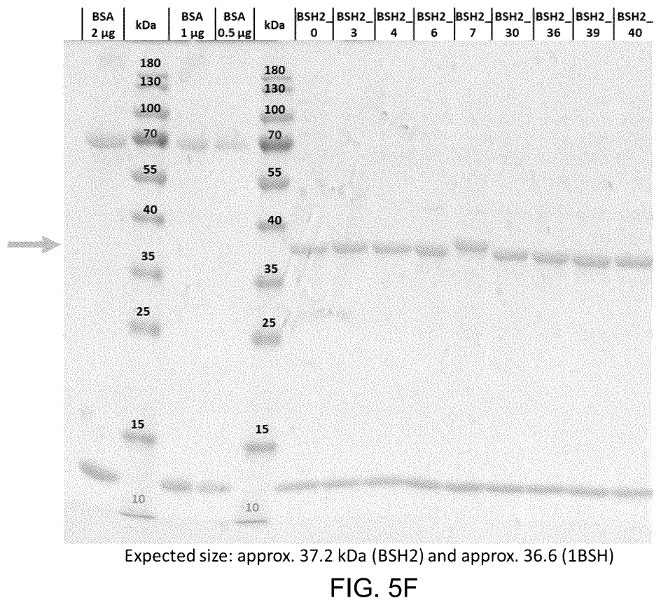

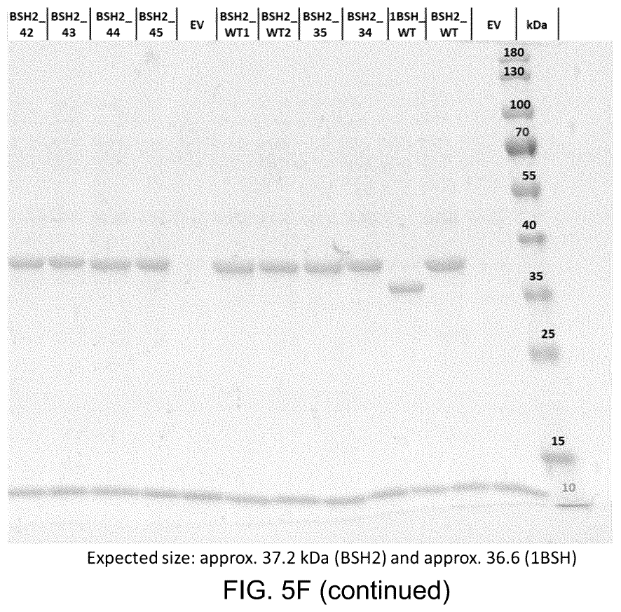

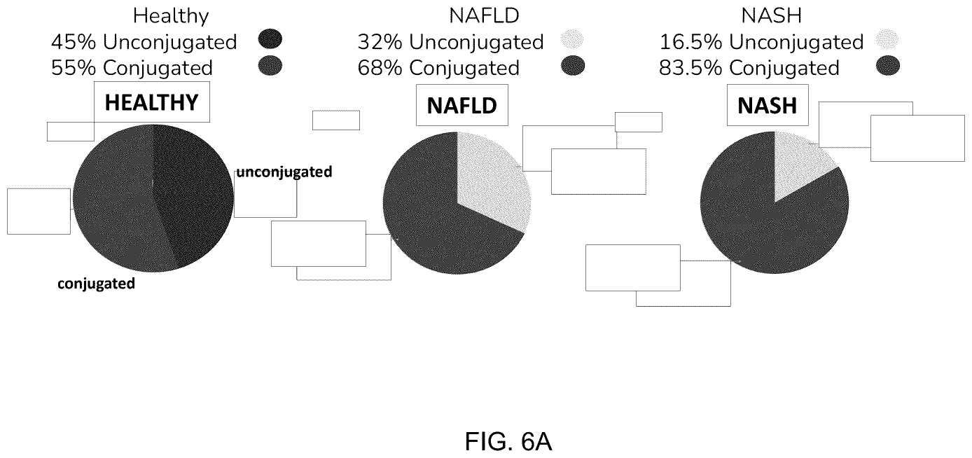

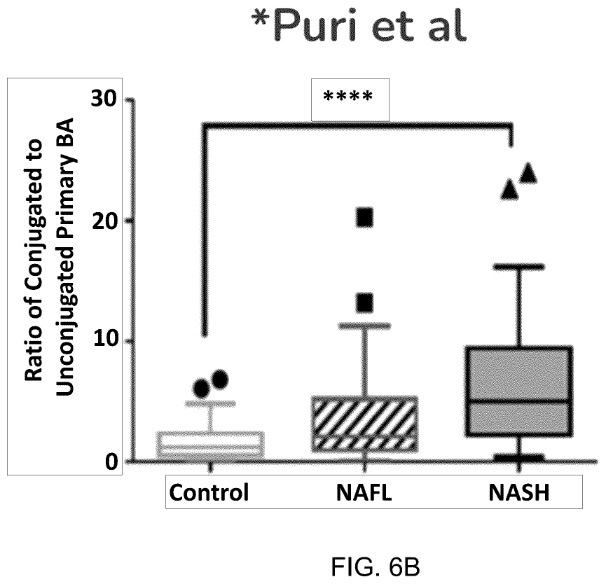

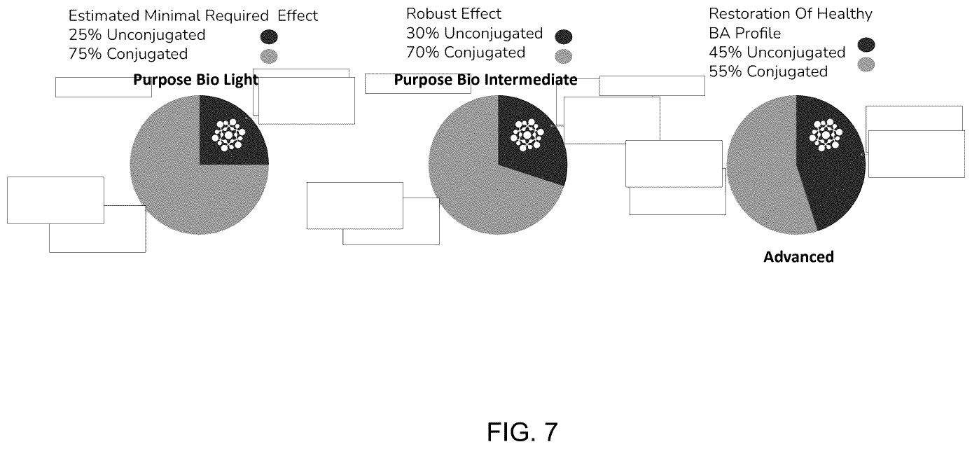

TABLE 1
Experimental naming convention and identity of modified BSH enzymes.

| | Naming | |
|---|---|---|
| | convention | |
| SEQ | used in | |
| ID NO | FIGS. 3 and 4 | Substitution(s) |
| 2 | BSH2_0 | C2S |
| 3 | BSH2_1 | Y8K |
| 4 | BSH2_2 | Y34N |
| 5 | BSH2_3 | I57V |
| 6 | BSH2_4 | Y64S |
| 7 | BSH2_5 | L127I |
| 8 | BSH2_6 | S136A |
| 9 | BSH2_7 | K189P |
| 10 | BSH2_8 | S206G |
| 11 | BSH2_9 | N292S |
| 12 | BSH2_10 | R323P |
| 13 | BSH2_11 | Y8T, Y56A |
| 14 | BSH2_12 | Y8Q, L127V |
| 15 | BSH2_13 | Y8K, N292S |
| 16 | BSH2_14 | I57V, L127I |
| 17 | BSH2_15 | Y34N, H47S |
| 18 | BSH2_16 | Y34N, N46K |
| 19 | BSH2_17 | Y34N, L127I |
| 20 | BSH2_18 | L127I, R323Q |
| 21 | BSH2_19 | S136A, N292S |
| 22 | BSH2_20 | N292S, R323T |
| 23 | BSH2_21 | Y8K, Y34N, Y56M |
| 24 | BSH2_22 | Y8Q, Y56T, L127I |
| 25 | BSH2_23 | Y34N, N46K, N292S |
| 26 | BSH2_24 | Y34N, H47S, N292S |
| 27 | BSH2_25 | Y34N, K189P, N292S |
| 28 | BSH2_26 | Y34N, I57V, N292S |
| 29 | BSH2_27 | Y34N, S136T, N292S |
| 30 | BSH2_28 | I57V, L127I, N292S |
| 31 | BSH2_29 | I57V, N292S, R323Q |
| 32 | BSH2_30 | L127I, K189P, N292R |
| 33 | BSH2_31 | Y8K, Y34N, L127I, N292S |
| 34 | BSH2_32 | Y8Q, Y34N, L127I, R323T |
| 35 | BSH2_33 | Y8K, Y56L, S206G, R207Q |
| 36 | BSH2_34 | Y8V, P81A, S206G, R207Q |
| 37 | BSH2_35 | Y34K, I57V, L127I, N292S |
| 38 | BSH2_36 | Y34S, N46K, L127I, N292S |
| 39 | BSH2_37 | Y34N, H47N, L127I, N292S |
| 40 | BSH2_38 | Y34N, S206G, R207Q, N292H |
| 41 | BSH2_39 | Y56T, I57V, L127I, N292S |
| 42 | BSH2_40 | K90E, L127I, N292S, R323E |
| 43 | BSH2_41 | Y34N, H47N, Y64F, Y176K, A221P |
| 44 | BSH2_42 | Y34N, N46K, H47N, Y64F, Y176K, A221L |
| 45 | BSH2_43 | Y8T, Y34N, Y56V, I57V, F99Y, L127I, S136A, K189N, N292S, R323L |
| 46 | BSH2_44 | I5V, Y8R, T9S, D11H, H12T, Y13F, R16M, L18Y, L20A, E21S, F22S, |
| | | Y24E, N25G, T27K, V28I, T29V, V30I, T31V, K33R, Y34K, F37L, |
| | | H38K, F39L, R40S, K43E, L45I, N46S, H47N, M54I, Y56K, I57V, |
| | | V58I, D60N, F61Y, Y64F, Y65K, D66M, T68C, S74A, G77A, L78I, |
| | | N79S, D82G, N83I, D85S, K87G, V89P, K90T, E91P, Y93K, D94E, |
| | | I96V, F99Y, F101L, W104Y, Q108R, A110G, S113D, R116L, I117E, |
| | | E120K, Q121T, I122L, L124I, L127V, N128D, E131A, L133H, S136K, |
| | | L138V, H139K, W140Y, Q145K, R146E, D147K, V151L, S153Q, |
| | | F161Y, V165T, N170G, F174Y, M178L, K189N, E190K, T195K, |
| | | A197S, A198P, E199S, L200I, E201K, Q204R, Y205I, R207S, A211N, |
| | | L218Y, S220D, A221L, R223K, V225I, K226R, A228S, T230V, K231R, |
| | | M232L, S234A, E240Y, S241D, S243Q, I244L, S245M, G252D, |
| | | E255R, Q256N, Q257V, R258P, C260T, E265G, E269L, I270R, I272L, |
| | | S274Q, S275T, C277I, N278D, K281R, Y284L, T287R, E290N, N292T, |
| | | Y300N, E302C, N303D, D305N, N307D, T308K, S311E, Y312F, |
| | | M315V, K316T, Q318P, N321Y, Y322E, R323L, N324K, Y325K |
| 47 | BSH2_45 | I5V, Y8K, T9S, D11S, H12S, Y13W, R16M, L18Y, L20A, E21S, F22S, |
| | | Y24E, T27E, V28I, T29V, V30I, T31V, K33R, Y34K, F37L, H38K, |
| | | F39L, R40S, K43T, L45I, N46D, H47E, Y49N, M54I, Y56E, V58R, |
| | | D60N, L63I, Y64F, Y65K, D66M, G77A, L78V, N79S, F80L, D82G, |
| | | N83I, D85S, K87G, V89K, K90T, Y93K, D94E, F99Y, F101L, W104Y, |
| | | Q108R, C109A, A110S, S113E, R116L, I117K, Q121N, I122L, L124I, |
| | | L127V, N128D, L133R, S136K, L138V, H139R, W140F, S143A, |
| | | Q145K, R146D, D147K, S148A, S153Q, F161Y, V165T, N170G, T173L, |
| | | F174Y, M178L, K189N, E190A, T195K, A198P, E199N, L200I, |
| | | E201K, Q204R, Y205I, R207A, M209D, A211N, L218K, S220D, A221L, |
| | | R223K, V225I, K226R, A228T, T230V, K231R, M232L, S234A, |
| | | S239T, S241D, S243Q, I244L, S245M, G252D, E255R, Q256D, |
| | | Q257V, R258P, C260T, H262W, E265G, E269L, I270R, I272L, S274Q, |
| | | S275V, C276A, C277I, K281T, I283K, Y284L, T287R, E290N, N292S, |
| | | T295N, A296M, Y300N, E302C, N303D, D305N, G306S, N307D, |
| | | T308K, Y312F, M315Q, Q318L, Q319D, N321K, Y322E, R323L, Y325K |

Referring now to FIGS. 3A, 3B, 4A, and 4B, the inventor has demonstrated that some substitutions resulted in distinct properties of the modified BSH enzymes of the instant disclosure. For example, BSH2_0, BSH2_4, BSH2_6, BSH2_7, BSH2_40, BSH2_42, BSH2_43, BSH2_44, BSH2_45, BSH2_34, and BSH2_35 showed increased GCA hydrolysis activity, compared to wild type BSH. BSH2_3, BSH2_4, BSH2_6, BSH2_7, BSH2_39, BSH2_40, BSH2_42, BSH2_43, BSH2_44, BSH2_45, BSH2_35, and BSH2_34 showed increased hydrolysis of GCDCA compared to wild type BSH.

As will be understood by one of skill in the art, the various compartments of human and non-human animal digestive systems have different levels of acidity/alkalinity. For example, the intraluminal pH is rapidly changed from highly acid in the stomach to about pH 6 in the duodenum. The pH gradually increases in the small intestine from pH 6 to about pH 7.4 in the terminal ileum. The pH drops to 5.7 in the caecum, but again gradually increases, reaching pH 6.7 in the rectum. Thus, the inventor contemplates that the disclosed modified BSH enzymes may have different or complementary applications owing to the possibility of differential pH-related activity.

The disclosed data also demonstrate that certain substitutions resulted in reduced or lack of activity in the modified BSH enzymes, e.g., the C2S substitution abolished enzyme activity. See, e.g., FIGS. 3A, 3B, 4A, and 4B.

The disclosed modified BSH enzymes may be produced in a recombinant system, e.g., the modified BSH enzymes may be expressed by a cell including, but not limited to, a mammalian cell, e.g., human embryonic kidney (HEK) cells, insect cells, fungal cells, e.g., yeast, bacterial cells, e.g., E. coli.

By way of example and not by way of limitation, in some embodiments, the modified BSH enzymes of the present disclosure may comprise one or more of the following additional modifications: codon optimization for better expression in a particular system; one or more post-translational modifications; one or more non-natural amino acids; one or more labels or detectable marker; one or more chemical moieties for linkage or encapsulation with a solid or semi-solid support such as a bead, a carrier molecule, or a target delivery molecule such as a protein or a virus.

### Polynucleotides

In an aspect of the current disclosure, polynucleotides are provided. In some embodiments, the polynucleotides comprise a sequence encoding the modified Christensenella minuta bile salt hydrolase (BSH) enzymes of the instant disclosure.

The polynucleotides may comprise one of SEQ ID NOs: 48-79. The polynucleotides may comprise one or more regulatory element. As used herein, “regulatory element” refers to a polynucleotide sequence that regulates the expression of another polynucleotide sequence, e.g., a promoter or enhancer.

As used herein, “operably linked” refers to a functional linkage between two or more sequences such that activity at or on one sequence affects activity at or on the other sequence(s). For example, an operable linkage between a polynucleotide of interest, e.g., a sequence encoding a modified BSH enzyme of the instant disclosure, and a regulatory element (e.g., a promoter) is a functional link that allows for expression of the polynucleotide of interest.

The promoters may be constitutive promoters or inducible promoters. A variety of promoters are known in the art, e.g., SEQ ID NOs: 48-52 or 58-61. See Table 2 for further details.

The promoter may be inducible by environmental conditions.

The promoter may be responsive to low oxygen or anaerobic conditions.

TABLE 2
Exemplary regulatory elements and other sequences.

| SEQ | | |
|---|---|---|
| ID NO | | Description |
| | Promoters | |
| 58 | BT1311 | constitutive, medium strong |
| 49 | P1TP + | constitutive but TetR repressible, starts at +1, |
| | GH023 | includes RBS |
| 50 | P2 | constitutive, strong, no RBS |
| 51 | P1 + | constitutive strong |
| | GH23 | |
| 52 | P1 | constitutive, strong, no RBS |
| | Ribosome binding sites (RBS) | |
| 53 | GH023 | |
| 54 | A21 | |
| 55 | B1 | |
| 56 | rpiL | |
| 57 | signal sequence from BfUbb | |
| | Promoter RBS combinations | |
| 58 | pPB001-T | Modified version of pPB001 (pNBU2-ermR with |
| | | integrase replaced by fragment of BT_1754 for |
| | | gene insertion to create fructose-insensitive |
| | | mutants) with additional rrnB T2 |
| | | terminator (-T). |
| 59 | pPB002 | BT1311 |
| 60 | pPB003 | GH023 |
| 61 | pPB004 | A21 |
| 62 | pPB005 | pNBU2-ermR plasmid backbone with BT_1311 |
| | | (rpoD) promoter and ribosome binding site. |
| | | Predicted to be constitutively active and |
| | | medium/high strength. |
| 63 | pPB006 | pNBU2-ermR plasmid backbone with BT_2163 |
| | | (rpiL) promoter and ribosome binding site. |
| | | Predicted to be constitutively active and |
| | | medium/high strength. |

As used herein, “selection marker” refers to any marker that can be used to select for expression of the polynucleotide, e.g., antibiotic resistance, auxotrophy, fluorescence, etc. Suitable selection markers are known in the art.

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be non-natural, modified, for example, by the addition of a chemical entity such as a carbohydrate group, a hydroxyl group, a phosphate group, a farnesyl group, an isofarnesyl group, a fatty acid group, a linker for conjugation, functionalization, labeled, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID NO, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The disclosed modified BSH enzymes may be delivered to a subject or host through any method, including but not limited to: live, static, chemical, nano particle, synthetic, etc., delivery methods.

Methods of Making Modified Christensenella minuta Bile Salt Hydrolase Enzymes

In an aspect of the current disclosure, methods of making the disclosed modified BSH enzymes are provided. In some embodiments, the methods of making the modified BSH enzymes comprise introducing a polynucleotide of the instant disclosure into a cell, wherein the cell expresses the polynucleotide. The methods may further comprise purifying, enriching, or isolating the modified BSH enzymes using methods known in the art including, but not limited to, chromatographic methods, e.g., liquid chromatography (LC), high performance liquid chromatography (HPLC), size exclusion chromatography (SEC), affinity chromatography, etc.

### Prebiotics

In an aspect of the current disclosure, prebiotics are provided. In some embodiments, the prebiotics comprise one or more of the disclosed modified BSH enzymes. The prebiotics may further comprise one or more additional ingredients to stabilize or preserve the modified BSH enzymes of the instant disclosure.

As used herein, “prebiotic” refers to a nondigestible food ingredient that promotes the growth of beneficial microorganisms in the digestive tract of an animal (for example, in the intestines).

The prebiotics are believed to modify the gut microbiome of a subject by increasing the amount of unconjugated bile acids. Further, increasing the unconjugated bile acids may improve the uptake of lipids from foods in livestock thereby improving the growth and health of livestock.

As discussed above, the modified BSH enzymes of the instant disclosure have distinct properties and may be selected or combined based on those properties, e.g., activity, selectivity, activity in a particular pH, to be included in a prebiotic composition. The prebiotics may comprise 1, 2, 3, 4, 5, 6, 7, 8, 9, 10, or more different modified enzyme types, i.e., modified BSH enzymes comprising distinct substitutions.

### Engineered Bacterial Cells

In an aspect of the current disclosure, engineered bacterial cells are provided. In some embodiments, the bacterial cells comprise a polynucleotide comprising a sequence encoding the disclosed modified BSH enzymes. The engineered bacteria are safe, well tolerated and can engraft in a host in order to improve the host's microbiome thereby extending a therapeutic benefit.

The disclosed bacterial cells are engineered to express the modified BSH enzymes. The engineered bacterial cells are believed to increase the amount of unconjugated bile acids in a subject, which is associated with reduced incidence of a variety of human and animal diseases and disorders including, but not limited to non-alcoholic fatty liver, non-alcoholic steatohepatitis, metabolic syndrome, and liver cancer.

The engineered bacterial cells may be, e.g., Christensenella minuta bacterial cells or any other suitable bacterial cell such as but not limited to one or more of the following non-limiting list of exemplary types of bacteria which may be used: Acidaminococcus spp., Actinomyces spp., Akkermansia muciniphila, Allobaculum spp., Anaerococcus spp., Anaerostipes spp., Bacteroides spp., Bacteroides thetaiotaomicron, Bacteroides vulgatus, Bacteroides thetaiotaomicron VPI-5482, Bacteroides fragilis NCTC-9343 Bacteroides ovatus, Bacteroides uniformis, Bacteroides eggerthii, Bacteroides Other, Bacteroides acidifaciens, Bacteroides coprophilus, Bacteroides fragilis, Bacteroides ovatus, Bacteroides uniformis, Barnesiellaceae spp., Bifidobacterium adolescentis, Bifidobacterium Other, Bifidobacterium spp., Bilophila spp., Blautia obeum, Blautia producta, Blautia Other, Blautia spp., Bulleidia spp., Catenibacterium spp., Chrisenella spp., Citrobacter spp., Clostridiaceae spp., Clostridiales Other, Clostridiales spp., Clostridium perfringens, Clostridium spp., Clostridium Other, Collinsella aerofaciens, Collinsella spp., Collinsella stercoris, Coprococcus catus, Coprococcus spp., Coriobacteriaceae spp., Desulfovibrionaceae spp., Dialister spp., Doreaformicigenerans, Dorea spp., Dorea Other, Eggerthella lenta, Enterobacteriaceae Other, Enterobacteriaceae spp., Enterococcus spp., Erysipelotrichaceae spp., Eubacterium biforme, Eubacterium biforme, Eubacterium dolichum, Eubacterium spp., Faecalibacterium prausnitzii, Fusobacterium spp., Gemellaceae spp., Haemophilus parainfluenzae, Haemophilus Other, Helicobacter spp., Helicobacter Lachnospiraceae Other, Lachnospiraceae spp., Lactobacillus reuteri, Lactobacillus mucosae, Lactobacillus zeae, Lactobacillus spp., Lactobacillaceae spp., Lactococcus spp., Leuconostocaceae spp., Megamonas spp., Megasphaera spp., Methanobrevibacter spp., Mitsuokella multacida, Mitsuokella spp., Mucispirillum schaedleri, Odoribacter spp., Oscillospira spp., Parabacteroides distasonis, Parabacteroides spp., Paraprevotella spp., Paraprevotellaceae spp., Parvimonas spp., Pediococcus spp., Pediococcus Other, Peptococcus spp., Peptoniphilus spp., Peptostreptococcus anaerobius, Peptostreptococcus Other, Phascolarctobacterium spp., Prevotella copri, Prevotella spp., Prevotella stercorea, Prevotellaceae, Proteus spp., Rikenellaceae spp., Roseburia faecis, Roseburia spp., Ruminococcaceae Other, Ruminococcaceae spp., Ruminococcus bromii, Ruminococcus gnavus, Ruminococcus spp., Ruminococcus Other, Ruminococcus torques, Slackia spp., S24-7 spp., SMB53 spp., Streptococcus anginosus, Streptococcus luteciae, Streptococcus spp., Streptococcus Other, Sutterella spp., Turicibacter spp., UC Bulleidia, UC Enterobacteriaceae, UC Faecalibacterium, UC Parabacteroides, UC Pediococcus, Varibaculum spp., Veillonella spp., Sutterella, Turicibacter, UC Clostridiales, UC Erysipelotrichaceae, UC Ruminococcaceae, Veillonella parvula, Veillonella spp., Veillonella dispar, s_Turicibacter_sp001543345:1, and Weissella.

Suitably, the engineered bacterial cell may be selected based on compatibility with a particular host. For example, in the case of an engineered bacterial cell designed to be administered or provided to a human, a bacterium that is a normal component of the human microbiome, e.g., of the gut microbiome, may be selected, e.g., C. minuta. In the case of selecting C. minuta as the bacterial cell, the native BSH enzyme in the cell may be disrupted or knocked out such that only expression of the modified BSH enzyme remains.

The bacterial cell may comprise a polynucleotide comprising a sequence encoding one or more of the modified BSH enzymes of the instant disclosure that is present and is integrated into the genome of the bacterial cell, or is present and expressed ectopically, i.e., not integrated into the bacterial cell genome. The bacterial cell may comprise multiple polynucleotides, each polynucleotide comprising nucleic acid sequence encoding one or more of the modified BSH enzymes of the present disclosure.

A polynucleotide comprising a sequence encoding the modified BSH enzymes of the instant disclosure may comprise a “heterologous promoter,” i.e., a promoter that is not native to the particular bacterial cell. For example, the bacterial cell may be a Christensenella minuta cell and the promoter may be from a different organism. In some embodiments, the heterologous promoter is functionally linked to the polynucleotide sequence encoding the modified BSH enzyme such that the engineered cell is capable of expressing the modified BSH enzyme.

### Probiotics

In an aspect of the current disclosure, probiotics are provided. In some embodiments, the probiotics comprise the engineered bacterial cells of the instant disclosure. The probiotics may further comprise one or more carrier or excipient.

As used herein, “probiotics” refers to live microorganisms that are intended to have health benefits when consumed or applied to the body. Probiotics may be used in nutraceutical compositions, which are also disclosed herein. Probiotics may also be referred to as live biotherapeutic products (LBPs).

The formulation of probiotics, e.g., to be ingested or delivered, is known in the art and is considered routine.

As discussed above, the provision of the disclosed modified enzymes, e.g., in the form of a probiotic comprising an engineered bacterial cell expressing the modified enzyme, is believed to be effective at increasing the amount of unconjugated bile acids in the gut of subject, e.g., human subjects or non-human animal subjects.

As the disclosed modified enzymes have different properties, e.g., activities and, potentially, specificity, potentially at different pHs, the probiotics may comprise more than one type of engineered bacterial cell. For example, the probiotics may comprise more than one species of bacterial cell. The probiotics may comprise more a first type of engineered bacterial cell comprising a particular modified BSH enzyme and a second type of engineered bacterial cell comprising a second different modified BSH enzyme. The different types of engineered bacterial cells may be selected to occupy different niches in the microbiome of the host (subject).

The disclosed probiotics may be used in methods, e.g., the disclosed probiotics may be provided or administered to a subject.

The disclosed probiotics may be used in methods of improving the yield of a livestock animal, the methods comprising providing an effective amount of a probiotic of the instant disclosure to improve the yield of the livestock.

The livestock animal may be, e.g., a ruminant, a pig, a bird, a fish, a crustacean, or a mollusk. The ruminant may be, e.g., a cow, a sheep, a goat, a deer, a buffalo, or a camelid.

### Animal Health

BSH enzymes in livestock play a crucial role in fat digestion by breaking down bile salts, which are essential for the absorption of dietary fats. This helps improve the nutritional efficiency and overall health and performance of the animals. Despite differences in lipid metabolism in humans vs. livestock, BSH remains an important enzyme in gut bacteria. Ruminants, such as cattle and sheep, have a unique digestive system that includes a specialized stomach chamber called the rumen. In the rumen, microbial fermentation of plant material occurs, and the resulting fatty acids and lipids can be modified by the gut microbiota before they are absorbed in the lower digestive tract. Modified BSH enzymes of the present disclosure produced by specific gut bacteria in ruminants may help in the hydrolysis of bile salts in this context, allowing the microbes to efficiently metabolize dietary lipids and produce volatile fatty acids that can be absorbed and used for energy. Non-ruminant livestock animals, like pigs and poultry, have a simpler digestive system without a rumen. Modified BSH enzymes of the present disclosure in these animals will primarily help with the digestion of dietary fats in the small intestine. They hydrolyze bile salts, making them less effective at emulsifying fats, which allows for the efficient digestion and absorption of these fats by the animal. Fatty acid profiles can also be affected. In ruminants, microbial fermentation and the involvement of modified BSH enzymes can lead to changes in the fatty acid profiles of the fats absorbed from the digestive tract. This may result in a higher proportion of volatile fatty acids (e.g., acetate, propionate, butyrate) that are used for energy and microbial protein synthesis in the ruminants themselves, as well as for the production of milk and meat.

In non-ruminant livestock, administration of the modified BSH enzymes may contribute to the efficient absorption of dietary fats, leading to the incorporation of a wide range of fatty acids into the animal's own tissues, which can affect the composition of fats in meat and eggs. The role of modified BSH enzyme in livestock can contribute to food security by improving the efficiency, sustainability, and resilience of animal agriculture, ultimately leading to increased availability and accessibility of animal-based protein sources for human consumption.

Modified BSH enzymes will contribute to better digestion and absorption of dietary fats in livestock. When animals can efficiently use the nutrients in their feed, it leads to improved feed conversion efficiency. This means that less feed is needed to produce a given amount of meat, milk, or eggs. Consequently, food resources are used more efficiently, and the production of animal-based protein is more sustainable.

Livestock that benefit from improved digestion and health due to administration of modified BSH enzymes of the instant disclosure are likely to exhibit better growth rates, higher milk production, or increased egg production. This increased productivity can lead to greater food output from the same number of animals, thereby contributing to food security by meeting the demand for animal-based protein.

Healthy livestock are more resistant to diseases, reducing the need for antibiotics and other pharmaceuticals. This, in turn, can reduce the risk of antibiotic-resistant pathogens emerging and affecting human health, which is a critical aspect of food safety and security.

By improving the efficiency of livestock production, administration of the modified BSH enzymes of the instant disclosure can help reduce the environmental footprint of animal agriculture. It can lead to reduced land and resource use, lower greenhouse gas emissions, and less water consumption per unit of food produced. This aligns with sustainable agriculture practices and contributes to long-term food security by ensuring the availability of resources for future generations.

Enhanced livestock productivity and health can help reduce food losses along the supply chain. Healthy animals are less likely to suffer from diseases that could lead to wastage, and efficient production means that less feed is wasted in producing the same amount of meat, milk, or eggs.

As described in this section “administration” of modified BSH enzymes may comprise administration of the enzyme as part of a therapeutic/nutritional composition, and/or administration of a cell expressing the enzyme as part of a therapeutic/nutritional composition.

### Aquaculture

Despite very different methods of lipid metabolism (fish have much simpler digestive systems than humans), modern aquaculture techniques involve feed containing high levels of fats and lipids. Addition of modified BSH enzymes of the instant disclosure, e.g., as a prebiotic or probiotic, to their diet may enhance their ability to digest and absorb these nutrients. This could lead to better growth rates and improved feed conversion efficiency, which is important for maximizing the productivity of aquaculture operations. By improving fat and lipid digestion, administration or provision of modified BSH enzymes of the instant disclosure can help fish and other aquatic organisms better utilize the nutrients present in their feed. This can lead to more efficient nutrient conversion into body mass, reducing the amount of waste produced and, in turn, potentially improving water quality in aquaculture systems.

The modified BSH enzymes of the present disclosure may allow for the use of alternative feed ingredients in aquaculture diets. Some feed ingredients, such as plant-based proteins and oils, can contain anti-nutritional factors like phytates that bind with minerals and reduce their bioavailability. The provision of administration of the modified BSH enzymes of the instant disclosure can help break down these compounds, making it possible to incorporate a wider variety of feed ingredients while maintaining nutritional value.

Improved nutrient utilization and growth efficiency can lead to reduced environmental impacts of aquaculture operations. If fish and other aquatic organisms grow more efficiently and produce less waste, there is less nutrient runoff and pollution in the surrounding aquatic ecosystems.

It is believed that the provision or administration of the modified BSH enzymes of the present disclosure may have a positive impact on the health of aquaculture species. Bile salts can have antimicrobial properties, and by breaking them down, the modified BSH enzymes can potentially reduce the prevalence of harmful pathogens in the gut. This could help reduce the incidence of diseases in aquaculture populations.

### Food Security

The provision or administration of the modified BSH enzymes to livestock may contribute to food security by improving the efficiency, sustainability, and resilience of animal agriculture, ultimately leading to increased availability and accessibility of animal-based protein sources for human consumption. Improved Nutrient Utilization: the modified BSH enzymes may contribute to better digestion and absorption of dietary fats in livestock. When animals can efficiently use the nutrients in their feed, it leads to improved feed conversion efficiency. This means that less feed is needed to produce a given amount of meat, milk, or eggs. Consequently, food resources are used more efficiently, and the production of animal-based protein is more sustainable.

Livestock that benefit from improved digestion and health due to administration of the modified BSH enzymes of the present disclosure are likely to exhibit better growth rates, higher milk production, or increased egg production. This increased productivity can lead to greater food output from the same number of animals, thereby contributing to food security by meeting the demand for animal-based protein. Enhanced Animal Health: Healthy livestock are more resistant to diseases, reducing the need for antibiotics and other pharmaceuticals. This, in turn, can reduce the risk of antibiotic-resistant pathogens emerging and affecting human health, which is a critical aspect of food safety and security.

By improving the efficiency of livestock production, the administration of provision of the modified BSH enzymes of the instant disclosure can help reduce the environmental footprint of animal agriculture. It can lead to reduced land and resource use, lower greenhouse gas emissions, and less water consumption per unit of food produced. This aligns with sustainable agriculture practices and contributes to long-term food security by ensuring the availability of resources for future generations. Enhanced livestock productivity and health can help reduce food losses along the supply chain. Healthy animals are less likely to suffer from diseases that could lead to wastage, and efficient production means that less feed is wasted in producing the same amount of meat, milk, or eggs.

### Bioremediation

The modified BSH enzymes of the present disclosure can be utilized in bioremediation processes to help break down and remove contaminants in water and soil. Some contaminants, like certain hydrophobic organic compounds, can be difficult to degrade naturally. Modified BSH enzymes of the present disclosure may aid in enhancing the degradation of these compounds by altering their interactions with bile salts and microbial communities.

### Kits

In an aspect of the disclosure, kits are provided. The kits may comprise any of the following components: nucleic acids encoding one or more of the modified BSH enzymes and optionally, cells for transformation or transfection with the nucleic acids, modified BSH enzymes, bacterial cells capable of expressing one or more of the modified BSH enzymes, pharmaceutical compositions comprising one or more of the BSH enzymes and/or cells expressing one or more of the modified BSH enzyme, medical foods comprising one or more of the BSH enzymes and/or cells expressing one or more of the modified BSH enzyme, etc. of the instant disclosure and may, optionally, comprise instructions for performing any of the disclosed methods.

### Pharmaceutical Compositions

In an aspect of the current disclosure, pharmaceutical compositions are provided. In some embodiments, the pharmaceutical compositions comprise the engineered bacterial cells of the instant disclosure, capable of expressing one or more modified BSH enzymes. In some embodiments, the pharmaceutical compositions comprise one or more of the modified BSH enzymes of the instant disclosure.

The pharmaceutical compositions may further comprise a pharmaceutically acceptable carrier or excipient, the formulation of which is routine and would be readily understood by one of skill in the art.

### Methods

In an aspect of the current disclosure, methods are provided. In some embodiments, the method comprise administering a pharmaceutical composition comprising one or more of the modified BSH enzymes described herein, and/or one or more cells expressing the modified BSH enzyme(s), of the instant disclosure to a subject in need thereof.

As used herein, a “subject in need thereof” or a “subject,” may refer to a vertebrate animal, e.g., livestock, e.g., cow, chicken, goat, sheep, llama, alpaca, camel, horse, donkey, fish, crustacean, mollusk, etc., a human, a companion animal, e.g., dog, cat, hamster, rat, chinchilla, ferret, etc., a laboratory animal, e.g., mouse, rat, rabbit, monkey, ape, etc. In some embodiments, the subject is a human. In some embodiments, the subject is a cat. A subject in need thereof may refer to a human suffering from NASH, NAFLD, Alzheimer's disease, Crohn's disease, a cholestatic disease, or a disease or disorder associated with metabolic syndrome.

The relative abundance of the bacterium C. minuta in the gut of humans is positively correlated with the lean host phenotype associated with a low BMI index. See, e.g., Ang, W. et al. A Keystone Gut Bacterium Christensenella minuta—A Potential Biotherapeutic Agent for Obesity and Associated Metabolic Diseases. Foods. 2023 July; 12(13): 2485; and Mazier, W. et al. A New Strain of Christensenella minuta as a Potential Biotherapy for Obesity and Associated Metabolic Diseases, Cells. 2021 April; 10(4): 823; which are incorporated by reference herein in their entireties. Further, modified C. minuta bacteria as a probiotic have been investigated (NCT04663139).

Fatty liver disease, also known as hepatic lipidosis, in cats typically occurs as a result of prolonged anorexia or starvation. When cats stop eating for extended periods, their bodies mobilize fat stores to meet energy needs, leading to an excessive accumulation of fat in the liver. This buildup interferes with liver function, impairing its ability to perform essential tasks such as detoxification and metabolism. Without prompt intervention, fatty liver disease can progress rapidly, leading to severe liver dysfunction, jaundice, and potentially liver failure. Treatment often involves aggressive nutritional support, including force-feeding or placement of a feeding tube to ensure adequate caloric intake. Veterinary care may also include supportive measures such as fluid therapy, vitamin supplementation, and medications to support liver function. Despite treatment efforts, the prognosis for cats with hepatic lipidosis can vary depending on the severity of the disease and the presence of underlying conditions. While many cats can recover with timely intervention, a significant percentage may succumb to complications associated with advanced liver disease. Prevention, early recognition and intervention are crucial for improving outcomes and minimizing the risk of mortality in cats with fatty liver disease. It is believed that the disclosed modified enzymes as pharmaceutical compositions or probiotics in food, may treat the signs or symptoms of feline hepatic lipidosis.

In some embodiments, the methods are methods of treating non-alcoholic fatty liver disease (NAFLD) in a subject in need thereof, the methods comprising administering a therapeutically effective amount of the pharmaceutical composition the instant disclosure to the subject to treat the NAFLD.

As used herein, a “therapeutically effective amount” or an “effective amount” refers to the amount or dose of the pharmaceutical composition that, upon single or multiple dose administration to the subject, provides the desired effect in the subject under diagnosis or treatment. For example, a therapeutically effective amount of the pharmaceutical compositions of the instant disclosure may comprise an amount effective to improve one or more sign or symptom associated with, e.g., NAFLD, non-alcoholic steatohepatitis (NASH), liver cancer, e.g., hepatocellular carcinoma (HCC), Alzheimer's disease, Crohn's disease, hyperlipidemia, dyslipidemia, hypercholesterolemia, obesity, cardiovascular disease, e.g., coronary artery disease, peripheral artery disease, carotid artery disease, heart failure, and stroke.

C. minuta has been reported to be absent from the microbiome of patients affected by Crohn's disease (CD) and have been documented to induce anti-inflammatory effects in human epithelial cells, supporting their potential as a negative regulator or treatment of CD. See, e.g., Relizani, K. et al. Selection of a novel strain of Christensenella minuta as a future biotherapy for Crohn's disease. Sci Rep. 2022; 12: 6017, which is incorporated by reference herein.

The disclosed pharmaceutical compositions may be used to treat cholestatic diseases, e.g., Primary biliary cirrhosis (PBC), primary sclerosing cholangitis (PSC), Alagille syndrome, Progressive familial intrahepatic cholestasis (PFIC), or biliary atresia.

Cholestatic disease refers to a group of conditions characterized by impaired bile flow from the liver, leading to the accumulation of bile acids and other substances in the liver and bloodstream. Bile acids are essential for the digestion and absorption of fats and fat-soluble vitamins in the intestines. In cholestatic diseases, bile acid dysfunction occurs due to a disruption in the normal bile flow process. This dysfunction can result in various symptoms, including jaundice, itching, fatigue, and in severe cases, liver damage.

Bile salt hydrolase (BSH) is an enzyme that plays a crucial role in bile acid metabolism. It catalyzes the hydrolysis of bile salts into bile acids and amino acids. In the context of cholestatic disease, where there is an accumulation of bile acids, BSH can serve as a therapeutic target. By enhancing the activity of BSH, excess bile salts can be broken down into bile acids, thereby reducing their toxic effects on liver cells and improving bile flow. This mechanism helps alleviate symptoms associated with cholestatic diseases and may even prevent further liver damage. Thus, the use of a novel BSH that has higher activity as a therapeutic intervention holds promise for the treatment of cholestatic diseases by restoring bile acid homeostasis and promoting liver health. See, e.g., Li, T. and Apte, U. Bile acid metabolism and signaling in cholestasis, inflammation and cancer Adv Pharmacol. 2015; 74: 263-302 and Zheng, J. et al Bile acid-mediated signaling in cholestatic liver diseases Cell & Bioscience volume 13, Article number: 77 (2023), each of which are incorporated by reference herein in their entireties.

Examples of cholestatic diseases may be treated using the disclosed pharmaceutical compositions, probiotics, prebiotics, and methods:

1. Primary Biliary Cholangitis (PBC): This is an autoimmune disease that primarily affects adults, particularly middle-aged women. It leads to inflammation and destruction of the small bile ducts within the liver.

2. Primary Sclerosing Cholangitis (PSC): Another autoimmune disorder, PSC causes inflammation and scarring (fibrosis) of the bile ducts, leading to obstruction and impaired bile flow. PSC can occur in both adults and children.

3. Biliary Atresia: This is a rare condition that affects newborns and infants. It involves the complete or partial blockage of bile ducts outside or inside the liver, leading to bile accumulation, liver damage, and eventually cirrhosis if left untreated.

4. Alagille Syndrome: This genetic disorder affects multiple organs, including the liver. Alagille syndrome is characterized by abnormalities in the bile ducts, leading to cholestasis and liver damage. It can present in infancy or childhood.

5. Progressive Familial Intrahepatic Cholestasis (PFIC): PFIC comprises a group of rare genetic disorders that result in impaired bile formation and cholestasis. It typically manifests in infancy or early childhood and can lead to progressive liver damage.

6. Benign Recurrent Intrahepatic Cholestasis (BRIC): BRIC is another rare genetic disorder characterized by intermittent episodes of cholestasis and jaundice. It usually presents in late childhood or adulthood and is generally less severe than PFIC.

As used herein, “treat” and grammatical variations thereof, refers to reducing or preventing at least one sign or symptom of a disease or disorder. For example, treating NAFLD may comprise reduction or prevention of weakness, loss of appetite, nausea, yellow skin and eyes (jaundice), itching, fluid buildup and swelling in the legs and abdomen, mental confusion, or gastrointestinal (GI) bleeding. With reference to HCC, treating may refer to reduction in abdominal discomfort or distention, jaundice, gastrointestinal hemorrhage, nausea or vomiting, persistent itching, or fever. With reference to obesity, treating may refer to a reduction in body fat percentage/composition. With reference to hyperlipidemia, hypercholesterolemia, treating may refer to normalization of blood lipid or cholesterol profiles, respectively. With reference to Alzheimer's disease (AD), treating may refer to reduction in confusion, aphasia, or other symptoms associated with AD. With reference to Crohn's disease treating may refer to reduction in frequency of bowel movements, reduction in pain associated with bowel movements, increase in body weight. With reference to cardiovascular disease, treating may refer to reducing numbness of the face, arm, or leg, especially on one side of the body, confusion, difficulty speaking or understanding speech; difficulty seeing with one or both eyes; difficulty walking, dizziness and/or loss of balance or coordination.

In some embodiments, the methods are methods of treating non-alcoholic steatohepatitis (NASH) in a subject in need thereof, the method comprising administering a therapeutically effective amount of the pharmaceutical composition of the instant disclosure to the subject to treat the NASH.

In some embodiments, the methods are methods of treating liver cancer in a subject in need thereof, the method comprising administering a therapeutically effective amount of the pharmaceutical composition of the instant disclosure to the subject to treat the liver cancer.

In some embodiments, the methods are methods of treating Alzheimer's disease in a subject in need thereof, the method comprising administering a therapeutically effective amount of the pharmaceutical composition of the instant disclosure to the subject to treat Alzheimer's disease.

### Liver Cancer

The disclosed engineered BSH enzymes have the potential to be harnessed as a therapeutic target for liver cancer due to its influence on bile acid metabolism and the gut-liver axis, which can be manipulated to disrupt pro-carcinogenic processes and promote anti-cancer mechanisms in the liver. The balance between conjugated and unconjugated bile acids in liver cancer is crucial, with excess conjugated bile acids potentially promoting inflammation and carcinogenesis, while high levels of conjugated bile acids may lead to liver cell damage and contribute to carcinogenesis. The disclosed enzymes promote an increase in unconjugated bile acids which is the therapeutic effect for treating and preventing liver cancer.

### Alzheimer's Disease

The role of lipid metabolism in Alzheimer's disease is an active area of research and there is evidence to suggest that lipid metabolism dysregulation may play a significant role in the development and progression of the disease. Alzheimer's disease is a complex neurodegenerative disorder characterized by the accumulation of amyloid plaques and tau tangles in the brain, as well as neuronal cell death and cognitive decline. Cholesterol is a type of lipid that is essential for brain function, as it is a major component of cell membranes and myelin, the protective sheath around nerve fibers. Abnormalities in cholesterol metabolism, such as high levels of LDL (“bad”) cholesterol or low levels of HDL (“good”) cholesterol, have been associated with an increased risk of Alzheimer's disease. It is believed that disruptions in cholesterol homeostasis may lead to the accumulation of amyloid beta plaques, a hallmark of Alzheimer's disease.

Lipid peroxidation, the oxidative degradation of lipids, can lead to the production of reactive oxygen species and inflammation. Oxidative stress and inflammation are believed to contribute to the pathogenesis of Alzheimer's disease. Lipid peroxidation can damage cellular membranes and disrupt neuronal function. Lipid rafts are specialized regions of cell membranes rich in cholesterol and certain types of lipids. They play a role in the processing of amyloid precursor protein (APP), which is cleaved to form amyloid beta, a peptide that accumulates in Alzheimer's disease. Alterations in lipid rafts may influence the cleavage of APP, potentially leading to increased amyloid beta production.

APOE is a protein involved in lipid transport in the brain. There are different isoforms of APOE, and the APOE ε4 allele is a major genetic risk factor for late-onset Alzheimer's disease. This allele has been associated with altered lipid metabolism in the brain and may affect the clearance of amyloid beta from the brain. The composition of lipids in the brain can impact membrane integrity and fluidity, which in turn affects neuronal function. Changes in the levels of specific lipids, such as sphingolipids and phospholipids, have been observed in the brains of individuals with Alzheimer's disease.

In some embodiments, the methods are methods of treating a disease or disorder associated with metabolic syndrome in a subject in need thereof, the method comprising administering a therapeutically effective amount of the pharmaceutical composition of the instant disclosure to the subject to treat the disease or disorder associated with metabolic syndrome. In some embodiments, the disease or disorder associated with metabolic syndrome is selected from the group consisting of: hyperlipidemia, hypercholesteremia, obesity, and cardiovascular disease. In some embodiments, the cardiovascular disease is selected from the group consisting of: coronary artery disease, peripheral artery disease, carotid artery disease, heart failure, and stroke.

As used herein, a “disease or disorder associated with metabolic syndrome” refers to any disease, disorder, or finding associated with metabolic syndrome. “Metabolic syndrome” refers to a cluster of conditions that increase the risk of heart disease, stroke, and diabetes. Metabolic syndrome includes high blood pressure, high blood sugar, excess body fat around the waist, and abnormal cholesterol levels.

The disclosed compositions and methods may be used to treat a sign or symptom of diabetes, e.g., type 2 diabetes, including, e.g., reducing fasting blood sugar.

### Bioremediation

BSH enzymes can be utilized in bioremediation processes to help break down and remove contaminants in water and soil. Some contaminants, like certain hydrophobic organic compounds, can be difficult to degrade naturally. The modified BSH enzymes of the present disclosure may aid in enhancing the degradation of these compounds by altering their interactions with bile salts and microbial communities.

### Food Products

In an aspect of the current disclosure, food products are provided. In some embodiments, the food products comprise lipids isolated from organisms by the methods of the instant disclosure, e.g., contacting an organism with the modified enzyme of the present disclosure and further comprising isolating, purifying, or extracting lipids from the organism.

The food products may take any form, e.g., a meat substitute, a food additive, etc.

### Medical Foods

In an aspect of the current disclosure, medical foods are provided. In some embodiments, the medical foods comprise the modified BSH enzymes, the prebiotics, bacterial cells, or probiotics of the instant disclosure.

As used herein, “medical food” refers to a food which is formulated to be consumed or administered enterally under the supervision of a physician and which is intended for the specific dietary management of a disease or condition for which distinctive nutritional requirements, based on recognized scientific principles, are established by medical evaluation.

The medical foods may be administered in any form including, but not limited to, powders, pills, yoghurt, capsules, tabs, gels, etc.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Illustrative Embodiments

1. A recombinant bacterial cell, wherein the recombinant bacterial cell harbors a heterologous BSH gene from the Christensenella minuta genome.

2. A recombinant bacterial cell from embodiment 1 wherein the expression of the BSH gene is controlled by a heterologous promoter.

3. The engineered bacteria from embodiments 1 and 2 wherein the promoter is an inducible promoter.

4. The engineered bacteria from embodiments 1 and 2 wherein the promoter is inducible by environmental conditions including those conditions specific to the gut of a mammal.

5. The engineered bacteria from embodiments 1-4 wherein the promoter is responsive to low oxygen or anaerobic conditions.

6. The engineered bacteria from embodiments 1-2 wherein the promoter is a constitutively active promoter.

7. The engineered bacteria from embodiments 1-6 further comprising a heterologous gene sequence encoding one or more BSH genes, including DSM33407.

8. The engineered bacterial cell from embodiments 1-7 wherein the BSH gene is directly or indirectly linked to an inducible promoter.

9. The engineered bacteria from embodiments 1-7 wherein the promoter is inducible by environmental factors, including those that are present in the gut of a mammal.

10. The engineered bacteria from embodiments 1-7 wherein the promoter is inducible by low oxygen or anaerobic conditions.

11. The engineered bacteria from embodiments 1-7 wherein the promoter is a constitutive promoter.

12. The engineered bacteria from embodiments 1-7 that contain a variant of said genes that have been further engineered to obtain increased enzymatic function, protein stability, a broader substrate specificity or any combination of those characteristics.

13. The engineered bacteria from embodiments 1-7 wherein the bacteria have been engineered to contain a combination of at least one BSH gene originally derived from Christensenella minuta and a second BSH allele from another bacteria commonly found in the mammalian gut flora. These microbiota associated bacterial strains include, but are not limited to Acidaminococcus spp., Actinomyces spp., Akkermansia spp, Akkermansia muciniphila, Allobaculum spp., Anaerococcus spp., Anaerostipes spp., Bacteroides spp., Bacteroides Other, Bacteroides acidifaciens, Bacteroides coprophilus, Bacteroides fragilis, Bacteroides ovatus, Bacteroides uniformis, Barnesiellaceae spp., Bifidobacterium adolescentis, Bifidobacterium Other, Bifidobacterium spp., Bilophila spp., Blautia obeum, Blautia producta, Blautia Other, Blautia spp., Bulleidia spp., Catenibacterium spp., Chrisenella spp., Christensenella minuta spp, Citrobacter spp., Clostridiaceae spp., Clostridiales Other, Clostridiales spp., Clostridium perfringens, Clostridium spp., Clostridium Other, Collinsella aerofaciens, Collinsella spp., Collinsella stercoris, Coprococcus catus, Coprococcus spp., Coriobacteriaceae spp., Desulfovibrionaceae spp., Dialister spp., Dorea formicigenerans, Dorea spp., Dorea Other, Eggerthella lenta, Enterobacteriaceae Other, Enterobacteriaceae spp., Enterococcus spp., Erysipelotrichaceae spp., Eubacterium biforme, Eubacterium biforme, Eubacterium dolichum, Eubacterium spp., Faecalibacterium prausnitzii, Fusobacterium spp., Gemellaceae spp., Haemophilus parainfluenzae, Haemophilus Other, Helicobacter spp., Helicobacter Lachnospiraceae Other, Lachnospiraceae spp., Lactobacillus reuteri, Lactobacillus mucosae, Lactobacillus zeae, Lactobacillus spp., Lactobacillaceae spp., Lactococcus spp., Leuconostocaceae spp., Megamonas spp., Megasphaera spp., Methanobrevibacter spp., Mitsuokella multacida, Mitsuokella spp., Mucispirillum schaedleri, Odoribacter spp., Oscillospira spp., Parabacteroides distasonis, Parabacteroides spp., Paraprevotella spp., Paraprevotellaceae spp., Parvimonas spp., Pediococcus spp., Pediococcus Other, Peptococcus spp., Peptoniphilus spp., Peptostreptococcus anaerobius, Peptostreptococcus Other, Phascolarctobacterium spp., Prevotella copri, Prevotella spp., Prevotella stercorea, Prevotellaceae, Proteus spp., Rikenellaceae spp., Roseburiafaecis, Roseburia spp., Ruminococcaceae Other, Ruminococcaceae spp., Ruminococcus bromii, Ruminococcus gnavus, Ruminococcus spp., Ruminococcus Other, Ruminococcus torques, Slackia spp., S24-7 spp., SMB53 spp., Streptococcus anginosus, Streptococcus luteciae, Streptococcus spp., Streptococcus Other, Sutterella spp., Turicibacter spp., UC Bulleidia, UC Enterobacteriaceae, UC Faecalibacterium, UC Parabacteroides, UC Pediococcus, Varibaculum spp., Veillonella spp., Sutterella, Turicibacter, UC Clostridiales, UC Erysipelotrichaceae, UC Ruminococcaceae, Veillonella parvula, Veillonella spp., Veillonella dispar, and Weissella.

14. The engineered bacterial cell from embodiments 1-13 wherein the BSH gene is derived from Christinsinella minuta genes and have been further engineered, using synthetic biology tools, to improve the half-life of the protein, the enzymatic activity per unit of protein, the bile salt substrate specificity, or any combination of the three characteristics listed herein.

15. The engineered bacterial cell from embodiments 1-14 wherein the BSH gene is directly or indirectly linked to an inducible promoter.

16. The engineered bacteria from embodiments 1-14 wherein the promoter is inducible by environmental factors, including those that are present in the gut of a mammal (human and/or non-human).

17. The engineered bacteria from embodiments 1-14 wherein the promoter is inducible by low oxygen or anaerobic conditions.

18. The engineered bacteria from embodiments 1-14 wherein the promoter is a constitutive promoter.

19. The engineered bacteria from embodiments 1-14 that contain a variant of said genes that have been further engineered to obtain increased enzymatic function, protein stability, a broader substrate specificity or any combination of those characteristics.

20. The engineered bacteria from embodiments 1-19 wherein the heterologous BSH containing genes originally derived from Christensenella minuta.

21. The engineered bacteria from embodiments 1-20 wherein the BSH gene is located in a plasmid within the bacterial cell.

22. The engineered bacteria from embodiments 1-20 wherein the BSH gene is integrated within the chromosome of the bacterial cell.

23. The engineered bacterial cell from embodiments 1-22 wherein engineered bacterial cell is a member of the genus Bacteroides, Bifidobacterium, Clostridium, Escherichia Coli, Lactobacillus, Lactococcus, or Turicibacter.

24. A pharmaceutical composition comprising the engineered bacterial cell of any of embodiments 1-23 and a pharmaceutically acceptable carrier.

25. A method for treating disease associated with bile salt dysregulation in a subject in need thereof, the method comprising administering the pharmaceutical composition of embodiment 24 to the subject.

26. A method for decreasing a level of bile salts in the gut of a subject, the method comprising administering the pharmaceutical composition of embodiment 24 to the subject, thereby decreasing the level of bile salts in the gut of subject.

27. A method for decreasing conjugated bile salts, unconjugated bile salts, or both conjugated bile salts and unconjugated bile salts in a subject, the method comprising administering the pharmaceutical composition of embodiment 24 to the subject, thereby decreasing the amount of conjugated bile salts, unconjugated bile salts, or both conjugated bile salts and unconjugated bile salts in the gut of the subject.

28. The method of embodiment 25, wherein the disorder associated with bile salt dysregulation is a metabolic disease or a liver fat storage disease.

29. The method of embodiment 25, wherein the disorder associated with bile salt dysregulation is a cardiovascular disease.

30. A method to improve the clinical signs or symptoms of a mammal who presents with NAFLD, the method comprising administering a bacterial cell comprising a BSH gene derived from Christensenella minuta strain DSM33407, or a functional equivalent of this gene that shares at least 90% identity with BSH gene derived from Christensenella minuta strain DSM33407.

31. The method of embodiment 30, wherein the bacterial cell expresses the BSH gene upon introduction to the gut of a mammal.

32. The method of any one of embodiments 25-31, wherein the bacterial cell is administered as part of a formulation suitable for oral delivery.

33. The method of any one of embodiments 1-32, wherein the subject is a mammal.

34. The method of embodiment 33, wherein the subject is a human.

35. The method of embodiment 33, wherein the subject is a dog, cat, horse, ferret, guinea pig, or hamster.

36. The method of embodiment 33, wherein the subject is a cow, goat, sheep, deer, yak, pig, donkey, reindeer, llama, alpaca, camel, or rabbit.

37. The method of embodiment 30, wherein the bacterial cell is an engineered Christensenella minuta.

38. A method of producing an improved animal product, the method comprising administering the pharmaceutical composition of embodiment 24 to a livestock animal; harvesting the livestock animal or a product from the livestock animal to produce an improved animal product.

39. The method of any one of embodiments 25-38, wherein administration comprises intravenous, intramuscular, subcutaneous, rectal, vaginal, or oral administration.

40. The method of embodiment 39, wherein administration comprises oral or rectal administration.

41. The method of embodiment 40, wherein administration comprises oral administration.

### EXAMPLES

The following Examples are illustrative and should not be interpreted to limit the scope of the claimed subject matter.

### Example 1—BSH Expression

### Introduction

### Aim

Aim of this protocol is to describe the complete set-up of experiments from E. coli cultivation to detection of the product after enzymatic reaction of cell lysate with substrate.

### Background Information

In previous experiments, it was confirmed that induction with 100 μM IPTG at OD₆₀₀~1.0 and subsequent expression at 37° C. for 20 h lead to expression of BSH.

### Materials

### Chemicals

Isopropyl β-d-1-thiogalactopyranoside (CAS: 367-93-1)

### Buffers Media

LB media

| | | |
|---|---|---|
| 1% | (w/v) | Tryptone |
| 1% | (w/v) | Sodium chloride |
| 0.5% | (w/v) | Yeast extract |

In ddH₂O
Consumables: Microtiter plate, 2.2 mL square V-bottom deep-well plate
Lab devices: Plate shaker, Centrifuge, Photo spectrometer

### Method

### Day 1 (Pre-Culture)

Microtiter plates were prepared with 145 μL LB Media including 50 μg/mL Kanamycin. The cryogenic cultures of the variants (BSH variants in E. coli C41 pLys) were thawed. After resuspension, each well was inoculated with 5 μL of a variant. MTP, containing 150 μL was sealed with a lid. MTP was incubated at 37° C. for 16 h under shaking at 900 rpm.

### Day 2 (Main-Culture)

To determine the growth of the cells, 5 μL of pre-culture were diluted 1:40 in 195 μL LB media. The absorption at 600 nm was measured. Pre-culture should be diluted to an OD₆₀₀~0.05 in LB for the main culture. 495 μL of LB incl. 50 μg/mL Kanamycin were added to each well of the deep-well plate. 5 μL of the pre-culture were added to the media. The inoculated media was grown at 37° C. under shaking at 900 rpm and 80% humidity until the bacterial cultures reached an OD₆₀₀~1.0, therefore the OD₆₀₀ was measured during cultivation. Once OD₆₀₀~1.0 was reached the cells were induced with 5 μL of 10 mM IPTG to a final concentration of 100 μM IPTG. After induction, the DWPs were sealed with a gas-permeable foil. Expression took place at 37° C. under shaking at 900 rpm and 80% humidity for 20 h.

### Harvest

After 20 h of expression the cells were harvested by centrifugation of the plates at 4,000 g for 10 min at 4° C. Afterwards the supernatant was discarded. The cell pellet was frozen at −20° C. for at least 1 h.

### Example 2—Taurine Assay

### Introduction

### Aim

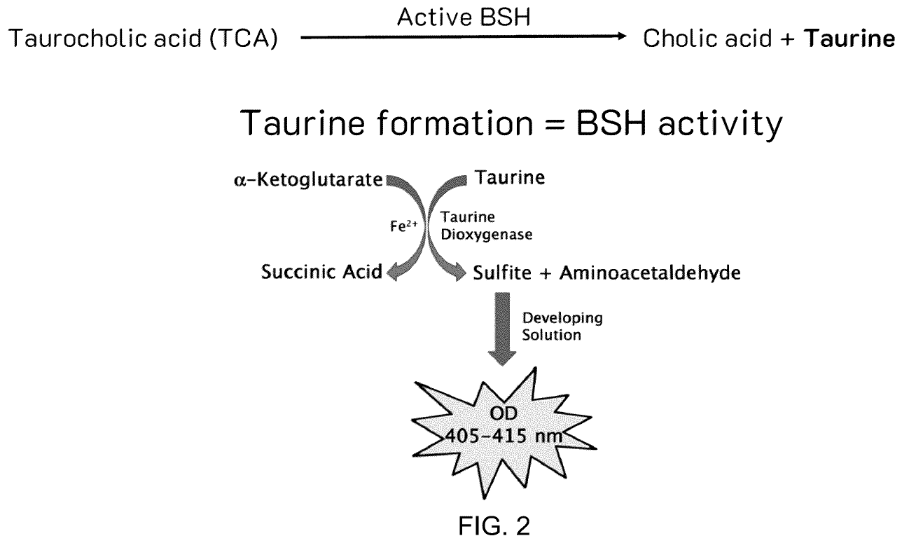

The aim of this prophetic experiment is to determine the taurine concentration after BSH assay on TCA. The taurine concentration indicates the activity of BSH. See, e.g., FIG. 2. The following protocol may be performed to detect the activity of the disclosed BSH enzymes in the hydrolysis of, e.g., taurocholic acid (TCA) to yield taurine and cholic acid.

### Background Information

Samples from Example 1 are used.

### Materials

### Chemicals

Taurine Assay Kit (MET-5071, cell biolabs)

### Buffers Media

TRIS/HCl buffer pH 9.0

| | |
|---|---|
| 200 mM | TRIS/HCl buffer |
| In ddH2O | |

Consumables: Clear MTP F-bottom.
Lab devices: Plate reader, Plate shaker

### Method

### Detailed Protocol

### Preparation of the Taurine Assay

The samples from the BSH assay are thawed. From the taurine assay kit taurine standards and taurine assay buffer are thawed at RT. The residual components are thawed on ice. Standards are prepared in duplicates with 0 μM, 15.6 μM, 31.3 μM, 62.5 μM, 125 μM, 250 μM, 500 μM and 1000 μM. The reaction mix is prepared according to the manual, see Table 3.

TABLE 3
Reaction mix for taurine assay kit.

| Component | Reaction mix [μL] |
|---|---|
| 10 × reagent A | 5 |
| 10 × reagent B | 5 |
| 100 × cofactor | 0.5 |
| Taurine dioxygenase | 5 |
| Assay buffer | 34.5 |

### Preparation of the Samples

On one hand, 50 μL of deproteinated sample from BSH assay at pH 7 are transferred into an MTP. On the other hand, 33 μL of deproteinated sample from BSH assay at pH 5 are transferred into an MTP and 17 μL of 200 mM TRIS/HCl buffer, pH 9 are added to these samples. Addition of pH 9 buffer to the pH 5 samples is necessary to reach a pH of ~7. All samples are performed as duplicates. One duplicate is directly measured with the taurine kit, whereas the other duplicate is quenched in order to determine the sample background (c.f. next section—Taurine assay).

### Taurine Assay

50 μL of the standard is transferred into an MTP. The reaction is started with 50 μL of reaction mix. Incubation is performed under shaking at 25° C. for 30 min. The reaction is stopped with 50 μL of stop solution and 50 μL of developing solution. For detection of sample background, the reaction is quenched with 50 μL of quenching solution and 50 μL of developing solution and incubated under shaking for 3 min at 25° C. The detection of absorbance (415 nm) is performed in a plate reader. The absorption of sample background (=quenched) is subtracted from the absorption of the sample, which is stopped with stop solution.

### Example 3—Glycine Assay

### Introduction

### Background Information

The samples from the BSH assay (Example 1) were thawed. From the glycine assay kit glycine standards, and glycine assay buffer were thawed at RT. The residual components were thawed on ice. Standards were prepared in duplicates with 0 μM, 1.56 μM, 3.13 μM, 6.25 μM, 12.5 μM, 25.0 μM, 50.0 μM and 100.0 μM. The reaction mix was prepared according to the manual.

### Materials

### Chemicals

Glycine assay kit (ab211100, abcam)

### Buffers Media (Table 4)

TRIS/HCl buffer pH 9.0

| | |
|---|---|
| 200 mM | TRIS/HCl buffer |
| In ddH₂O | |

Consumables: Black MTP, Lab devices, Plate reader, Plate shaker.

### Method

Samples were thawed. Components of the glycine assay kit were thawed with GLY Assay buffer at RT, the residual components of the kit on ice. 1 mM standard was prepared by diluting 5 μL of the 100 mM GLY Standard with 495 μL ddH₂O. 50 μM standard, by diluting 50 μL of the 1 mM GLY Standard with 950 μL ddH₂O. As standards 0 μM, 2 μM, 4 μM, 6 μM, 8 μM and 10 μM glycine were used. For one reaction the reaction mix was prepared according the manual, see to Table 5.

TABLE 5
Reaction mix for glycine assay kit.

| Component | Reaction mix [μL] |
|---|---|
| Fluorometric probe | 0.5 |
| HRP | 0.1 |
| Glycine oxidase | 25 |
| Assay buffer | 24.4 |

### Preparation of the Samples

The samples with pH 7 were diluted 1:3 in assay buffer. Samples with pH 5 were diluted 2:3 in buffer at pH 9 and then diluted further 1:2 in assay buffer.

### Glycine Assay

50 μL of standard were transferred into a black MTP. 50 μL of 1:3 diluted samples were transferred into a black MTP. The reaction was started with 50 μL of reaction mix. Incubation was performed protected from light under shaking at 900 rpm at 37° C. for 30-60 min. The fluorescence was measured at Ex/Em 560/587 after 60 min.

### Example 4—Treatment of Non-Alcoholic Fatty Liver Disease

In one example, a subject suffering from non-alcoholic fatty liver disease (NAFLD) is administered a therapeutically effective amount of the disclosed compositions, e.g., pharmaceutical compositions, prebiotics, or probiotics. The compositions may suitably be administered by any route that is indicated by the particular treatment needs of the subject, e.g., oral or intrarectal routes. Signs and symptoms of the NAFLD may be reduced by the administration of the compositions. Treatment may be administered daily, every other day, every third day, or on a schedule as determined by the patient's progress, pursuant to a physician's decision. It is anticipated that the subject will experience an increase in body mass, appetite, or amelioration of nausea, vomiting, abdominal pain, or itching or other metrics associated with reduction in signs or symptoms of NAFLD, as compared to an untreated subject. Methods of measuring reductions in signs and symptoms of NAFLD are known in the art.

### Example 5—Treatment of Non-Alcoholic Steatohepatitis

In one example, a subject suffering from non-alcoholic steatohepatitis (NASH) is administered a therapeutically effective amount of the disclosed compositions, e.g., pharmaceutical compositions, prebiotics, or probiotics. The compositions may suitably be administered by any route that is indicated by the particular treatment needs of the subject, e.g., oral or intrarectal routes. Signs and symptoms of the NASH may be reduced by the administration of the compositions. Treatment may be administered daily, every other day, every third day, or on a schedule as determined by the patient's progress, pursuant to a physician's decision. It is anticipated that the subject will experience an increase in body mass, appetite, or amelioration of nausea, vomiting, abdominal pain, or itching or other metrics associated with reduction in signs or symptoms of NASH, as compared to an untreated subject. Methods of measuring reductions in signs and symptoms of NASH are known in the art.

TABLE 6
Polynucleotide sequences encoding
the disclosed modified enzymes.

| SEQ ID NO: | Enzyme index No. see Table 1. |
|---|---|
| 64 | WT (codon optimized for E. coli) |
| 65 | 0 |
| 66 | 3 |
| 67 | 4 |
| 68 | 6 |
| 69 | 7 |
| 70 | 30 |
| 71 | 34 |
| 72 | 35 |
| 73 | 36 |
| 74 | 39 |
| 75 | 40 |
| 76 | 42 |
| 77 | 43 |
| 78 | 44 |
| 79 | 45 |

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A modified Christensenella minuta (C. minuta) bile salt hydrolase (BSH) enzyme at least 90% identical to SEQ ID NO: 1 and comprising one or more amino acid substitution at position C2, Y8, Y34, N46, H47, Y56, I57, Y64, P81, K90, F99, L127, S136, Y176, K189, S206, R207, A221, N292, or R323 relative to SEQ ID NO: 1.

2. The modified enzyme of claim 1, wherein the one or more substitution comprises C2S, Y8K, Y8T, Y8Q, Y8V, Y8T, Y34N, Y34S, N46K, H47S, H47N, Y56A, Y56M, Y56T, Y56L, Y56V, I57V, Y64S, Y64F, P81A, K90E, F99Y, L127I, L127V, S136A, S136T, Y176K, K189P, K189N, S206G, R207Q, A221P, A221L, N292S, N292R, N292H, R323P, R323T, R323E, R323L, or R323Q.

3. The modified enzyme of claim 1, wherein the one or more substitution comprises Y8V, Y34K, I57V, Y64S, P81A, K90E, L127I, S136A, K189P, K189N, S206G, R207Q, N292S, R323L, or R323E.

4. The modified enzyme of claim 1, wherein the one or more substitution comprises I57V.

5. The modified enzyme of claim 1, wherein the one or more substitution comprises Y64S.

6. The modified enzyme of claim 1, wherein the one or more substitution comprises S136A.

7. The modified enzyme of claim 1, wherein the one or more substitution comprises K189P.

8. The modified enzyme of claim 1, wherein the one or more substitution comprises Y8V, P81A, S206G, and R207Q.

9. The modified enzyme of claim 1, wherein the one or more substitution comprises Y34K, I57V, L127I, and N292S.

10. The modified enzyme of claim 1, wherein the one or more substitution comprises Y56T, I57V, L127I, and N292S.

11. The modified enzyme of claim 1, wherein the one or more substitution comprises K90E, L127I, N292S, and R323E.

12. The modified enzyme of claim 1, wherein the one or more substitution comprises Y34N, N46K, H47N, Y64F, Y176K, and A221L.

13. The modified enzyme of claim 1, wherein the one or more substitution comprises Y8T, Y34N, Y56V, I57V, F99Y, L127I, S136A, K189N, N292S, and R323L.

14. The modified enzyme of claim 1, wherein the modified enzyme comprises one of SEQ ID NOs: 2-47.

15. The modified enzyme of claim 1, wherein the modified enzyme comprises a sequence with at least 90% identity to SEQ ID NO: 5 and wherein the modified enzyme has the substitution I57V, with regard to SEQ ID NO: 1, optionally, wherein the modified enzyme comprises or consists of SEQ ID NO: 5.

16. The modified enzyme of claim 1, wherein the modified enzyme comprises a sequence with at least 90% identity to SEQ ID NO: 6 and wherein the modified enzyme has the substitution Y64S, with regard to SEQ ID NO: 1, optionally, wherein the modified enzyme comprises or consists of SEQ ID NO: 6.

17. The modified enzyme of claim 1, wherein the modified enzyme comprises a sequence with at least 90% identity to SEQ ID NO: 8 and wherein the modified enzyme has the substitution S136A, with regard to SEQ ID NO: 1, optionally, wherein the modified enzyme comprises or consists of SEQ ID NO: 8.

18. The modified enzyme of claim 1, wherein the modified enzyme comprises a sequence with at least 90% identity to SEQ ID NO: 9 and wherein the modified enzyme has the substitution K189P, with regard to SEQ ID NO: 1, optionally, wherein the modified enzyme comprises or consists of SEQ ID NO: 9.

19. The modified enzyme of claim 1, wherein the modified enzyme comprises a sequence with at least 90% identity to SEQ ID NO: 36 and wherein the modified enzyme has the substitutions Y8V, P81A, S206G, and R207Q, with regard to SEQ ID NO: 1, optionally, wherein the modified enzyme comprises or consists of SEQ ID NO: 36.

20. The modified enzyme of claim 1, wherein the modified enzyme comprises a sequence with at least 90% identity to SEQ ID NO: 37 and wherein the modified enzyme has the substitutions Y34K, I57V, L127I, and N292S, with regard to SEQ ID NO: 1, optionally, wherein the modified enzyme comprises or consists of SEQ ID NO: 37.

21. The modified enzyme of claim 1, wherein the modified enzyme comprises a sequence with at least 90% identity to SEQ ID NO: 41 and wherein the modified enzyme has the substitutions Y56T, I57V, L127I, and N292S, with regard to SEQ ID NO: 1, optionally, wherein the modified enzyme comprises or consists of SEQ ID NO: 41.

22. The modified enzyme of claim 1, wherein the modified enzyme comprises a sequence with at least 90% identity to SEQ ID NO: 42 and wherein the modified enzyme has the substitutions K90E, L127I, N292S, and R323E, with regard to SEQ ID NO: 1, optionally, wherein the modified enzyme comprises or consists of SEQ ID NO 42.

23. The modified enzyme of claim 1, wherein the modified enzyme comprises a sequence with at least 90% identity to SEQ ID NO: 43 and wherein the modified enzyme has the substitutions Y34N, H47N, Y64F, Y176K, A221P, with regard to SEQ ID NO: 1, optionally, wherein the modified enzyme comprises or consists of SEQ ID NO 43.

24. The modified enzyme of claim 1, wherein the modified enzyme comprises a sequence with at least 90% identity to SEQ ID NO: 44 and wherein the modified enzyme has the substitutions Y34N, N46K, H47N, Y64F, Y176K, and A221L, with regard to SEQ ID NO: 1, optionally, wherein the modified enzyme comprises or consists of SEQ ID NO 44.

25. The modified enzyme of claim 1, wherein the modified enzyme comprises a sequence with at least 90% identity to SEQ ID NO: 45 and wherein the modified enzyme has the substitutions Y8T, Y34N, Y56V, I57V, F99Y, L127I, S136A, K189N, N292S, and R323L, with regard to SEQ ID NO: 1, optionally, wherein the modified enzyme comprises or consists of SEQ ID NO 45.

26. A modified bile salt hydrolase (BSH) enzyme comprising amino acid substitutions at position I5V, Y8R, T9S, D11H, H12T, YT3F, R16M, L18Y, L20A, E21S, F22S, Y24E, N25G, T27K, V28I, T29V, V30I, T3TV, K33R, Y34K, F37L, H38K, F39L, R40S, K43E, L45I, N46S, H47N, M54I, Y56K, I57V, V58I, D60N, F61Y, Y64F, Y65K, D66M, T68C, S74A, G77A, L78I, N79S, D82G, N83I, D85S, K87G, V89P, K90T, E91P, Y93K, D94E, 196V, F99Y, F101L, W104Y, Q108R, A110G, S113D, R116L, I117E, E120K, Q121T, I122L, L124I, L127V, N128D, E131A, L133H, S136K, L138V, H139K, W140Y, Q145K, R146E, D147K, V151L, S153Q, F161Y, V165T, N170G, F174Y, M178L, K189N, E190K, T195K, A197S, A198P, E199S, L200I, E201K, Q204R, Y205I, R207S, A211N, L218Y, S220D, A221L, R223K, V225I, K226R, A228S, T230V, K231R, M232L, S234A, E240Y, S241D, S243Q, I244L, S245M, G252D, E255R, Q256N, Q257V, R258P, C260T, E265G, E269L, I270R, I272L, S274Q, S275T, C277I, N278D, K281R, Y284L, T287R, E290N, N292T, Y300N, E302C, N303D, D305N, N307D, T308K, S311E, Y312F, M315V, K316T, Q318P, N321Y, Y322E, R323L, N324K, and Y325K, relative to SEQ ID NO: 1.

27. A modified bile salt hydrolase (BSH) enzyme comprising amino acid substitutions at position I5V, Y8K, T9S, D11S, H12S, Y13W, R16M, L18Y, L20A, E21S, F22S, Y24E, T27E, V28I, T29V, V30I, T31V, K33R, Y34K, F37L, H38K, F39L, R40S, K43T, L45I, N46D, H47E, Y49N, M54I, Y56E, V58R, D60N, L63I, Y64F, Y65K, D66M, G77A, L78V, N79S, F80L, D82G, N83I, D85S, K87G, V89K, K90T, Y93K, D94E, F99Y, F101L, W104Y, Q108R, C109A, A110S, S113E, R116L, I117K, Q121N, I122L, L124I, L127V, N128D, L133R, S136K, L138V, H139R, W140F, S143A, Q145K, R146D, D147K, S148A, S153Q, F161Y, V165T, N170G, T173L, F174Y, M178L, K189N, E190A, T195K, A198P, E199N, L200I, E201K, Q204R, Y205I, R207A, M209D, A211N, L218K, S220D, A221L, R223K, V225I, K226R, A228T, T230V, K231R, M232L, S234A, S239T, S241D, S243Q, I244L, S245M, G252D, E255R, Q256D, Q257V, R258P, C260T, H262W, E265G, E269L, I270R, I272L, S274Q, S275V, C276A, C277I, K281T, I283K, Y284L, T287R, E290N, N292S, T295N, A296M, Y300N, E302C, N303D, D305N, G306S, N307D, T308K, Y312F, M315Q, Q318L, Q319D, N321K, Y322E, R323L, and Y325K, relative to SEQ ID NO: 1.

28. A modified bile salt hydrolase (BSH) enzyme comprising or consisting of one of SEQ ID NOs: 46 or 47.

29. The modified enzyme of claim 1, wherein the modified enzyme has increased activity compared to wild type C. minuta BSH at about pH 5.

30. The modified enzyme of claim 1, wherein the modified enzyme has increased activity compared to wild type C. minuta BSH at about pH 7.

31. A polynucleotide comprising a sequence encoding the modified Christensenella minuta bile salt hydrolase (BSH) enzyme of claim 1.

32. The polynucleotide of claim 31, wherein the polynucleotide comprises one of SEQ ID NOs: 48-79.

33. The polynucleotide of claim 31, further comprising one or more regulatory elements, wherein the one or more regulatory elements is operably linked to the sequence encoding the modified enzyme.

34. The polynucleotide of claim 31, wherein the one or more regulatory elements comprises a promoter, and/or enhancer.

35. The polynucleotide of claim 31, further comprising a selection marker.

36. The polynucleotide of claim 33, wherein the one or more regulatory elements is a promoter and the promoter is a constitutive promoter.

37. The polynucleotide of claim 33, wherein the one or more regulatory elements is a promoter and the promoter is an inducible promoter.

38. The polynucleotide of claim 37, wherein the promoter is inducible by environmental conditions or is responsive to low oxygen or anaerobic conditions.

39. The polynucleotide of claim 37, wherein the polynucleotide comprises one of SEQ ID NOs: 104-109.

40. A prebiotic comprising the modified Christensenella minuta bile salt hydrolase (BSH) enzyme of claim 1.

41. A pharmaceutical composition comprising the modified Christensenella minuta bile salt hydrolase (BSH) enzyme of claim 1.

42. An engineered bacterial cell comprising the polynucleotide of claim 31.

43. The engineered bacterial cell of claim 42, wherein the expression of the polynucleotide is operably linked to an exogenous promoter not found in the natural Christensenella minuta genome.

44. The engineered bacterial cell of claim 42, wherein the engineered bacterial cell is selected from the group consisting of: Acidaminococcus spp., Actinomyces spp., Akkermansia muciniphila, Allobaculum spp., Anaerococcus spp., Anaerostipes spp., Bacteroides spp., Bacteroides Other, Bacteroides acidifaciens, Bacteroides coprophilus, Bacteroides fragilis, Bacteroides ovatus, Bacteroides uniformis, Barnesiellaceae spp., Bifidobacterium adolescentis, Bifidobacterium Other, Bifidobacterium spp., Bilophila spp., Blautia obeum, Blautia producta, Blautia Other, Blautia spp., Bulleidia spp., Catenibacterium spp., Chrisenella spp., Citrobacter spp., Clostridiaceae spp., Clostridiales Other, Clostridiales spp., Clostridium perfringens, Clostridium spp., Clostridium Other, Collinsella aerofaciens, Collinsella spp., Collinsella stercoris, Coprococcus catus, Coprococcus spp., Coriobacteriaceae spp., Desulfovibrionaceae spp., Dialister spp., Dorea formicigenerans, Dorea spp., Dorea Other, Eggerthella lenta, Enterobacteriaceae Other, Enterobacteriaceae spp., Enterococcus spp., Erysipelotrichaceae spp., Eubacterium biforme, Eubacterium biforme, Eubacterium dolichum, Eubacterium spp., Faecalibacterium prausnitzii, Fusobacterium spp., Gemellaceae spp., Haemophilus parainfluenzae, Haemophilus Other, Helicobacter spp., Helicobacter Lachnospiraceae Other, Lachnospiraceae spp., Lactobacillus reuteri, Lactobacillus mucosae, Lactobacillus zeae, Lactobacillus spp., Lactobacillaceae spp., Lactococcus spp., Leuconostocaceae spp., Megamonas spp., Megasphaera spp., Methanobrevibacter spp., Mitsuokella multacida, Mitsuokella spp., Mucispirillum schaedleri, Odoribacter spp., Oscillospira spp., Parabacteroides distasonis, Parabacteroides spp., Paraprevotella spp., Paraprevotellaceae spp., Parvimonas spp., Pediococcus spp., Pediococcus Other, Peptococcus spp., Peptoniphilus spp., Peptostreptococcus anaerobius, Peptostreptococcus Other, Phascolarctobacterium spp., Prevotella copri, Prevotella spp., Prevotella stercorea, Prevotellaceae, Proteus spp., Rikenellaceae spp., Roseburia faecis, Roseburia spp., Ruminococcaceae Other, Ruminococcaceae spp., Ruminococcus bromii, Ruminococcus gnavus, Ruminococcus spp., Ruminococcus Other, Ruminococcus torques, Slackia spp., S24-7 spp., SMB53 spp., Streptococcus anginosus, Streptococcus luteciae, Streptococcus spp., Streptococcus Other, Sutterella spp., Turicibacter spp., UC Bulleidia, UC Enterobacteriaceae, UC Faecalibacterium, UC Parabacteroides, UC Pediococcus, Varibaculum spp., Veillonella spp., Sutterella, Turicibacter, UC Clostridiales, UC Erysipelotrichaceae, UC Ruminococcaceae, Veillonella parvula, Veillonella spp., Veillonella dispar, and Weissella.

45. The bacterial cell of claim 42, wherein the polynucleotide is integrated into a genome of the bacterial cell.

46. A probiotic composition comprising the engineered bacterial cell of claim 42.

47. A pharmaceutical composition comprising the engineered bacterial cell of claim 42 or the modified BSH enzyme of claim 1.

48. A method comprising administering the pharmaceutical composition of claim 47 to a subject in need thereof.

49. The method of claim 48, wherein the subject in need thereof is suffering from one or more of: non-alcoholic fatty liver disease (NAFLD), non-alcoholic fatty liver disease (NAFLD), a disease or disorder associated with metabolic syndrome, or cardiovascular disease.

50. A method of treating non-alcoholic fatty liver disease (NAFLD) in a subject in need thereof, the method comprising administering a therapeutically effective amount of the pharmaceutical composition of claim 47 to the subject to treat the NAFLD.

51. A method of treating non-alcoholic steatohepatitis (NASH) in a subject in need thereof, the method comprising administering a therapeutically effective amount of the pharmaceutical composition of claim 47 to the subject to treat the NASH.

52. A method of treating liver cancer in a subject in need thereof, the method comprising administering a therapeutically effective amount of the pharmaceutical composition of claim 47 to the subject to treat the liver cancer.

53. A method of treating a cholestatic disease in a subject in need thereof, the method comprising administering a therapeutically effective amount of the pharmaceutical composition of claim 47 to the subject to treat the cholestatic disease in the subject.

54. A method of treating Alzheimer's disease in a subject in need thereof, the method comprising administering a therapeutically effective amount of the pharmaceutical composition of claim 47 to the subject to treat Alzheimer's disease.

55. A method of treating Crohn's disease in a subject in need thereof, the method comprising administering a therapeutically effective amount of the pharmaceutical composition of claim 47 to the subject to treat Crohn's disease in the subject.

56. A method of treating a disease or disorder associated with metabolic syndrome in a subject in need thereof, the method comprising administering a therapeutically effective amount of the pharmaceutical composition of claim 47 to the subject to treat the disease or disorder associated with metabolic syndrome.

57. The method of claim 47, wherein the disease or disorder associated with metabolic syndrome is selected from the group consisting of hyperlipidemia, hypercholesteremia, obesity, and cardiovascular disease.

58. The method of claim 57, wherein the cardiovascular disease is selected from the group consisting of coronary artery disease, peripheral artery disease, carotid artery disease, heart failure, and stroke.

59. The method of any one of claims 48-58, wherein the subject in need thereof is an animal.

60. The method of claim 59, wherein the subject in need thereof is a mammal.

61. The method of claim 59, wherein the subject in need thereof is a dog, cat, cow, sheep, goat, chicken, turkey, pig, fish, crustacean, or mollusk.

62. The method of claim 59, wherein the subject in need thereof is a human subject.

63. The method of claim 59, wherein the subject in need thereof is a cat and wherein the cat is suffering from feline hepatic lipidosis.

64. A method comprising providing the probiotic of claim 46 or the prebiotic of claim 40 to a subject.

65. A method of improving the yield of a livestock animal, the method comprising providing an effective amount of the probiotic of claim 46 to improve the yield of the livestock.

66. The method of claim 65, wherein the yield of the livestock animal is improved as compared to a member of the same species of livestock that has not been provided the probiotic.

67. The method of claim 66, wherein the livestock animal is a ruminant, a pig, a bird, a fish, a crustacean, or a mollusk.

68. The method of claim 67, wherein the ruminant is selected from a cow, a sheep, a goat, a deer, a buffalo, or a camelid.

69. The food product of claim 68, wherein the lipids are fatty acids.

70. A medical food comprising the modified BSH enzyme of claim 1, the prebiotic of claim 40, or the bacterial cell of claim 42.

71. A method of making a modified bile salt hydrolase (BSH) enzyme, the method comprising introducing the polynucleotide of claim 31 into a cell, thereby causing the cell to express the modified BSH enzyme.

72. The method of claim 71, further comprising enriching, purifying, or isolating the modified BSH enzyme.

73. A modified Christensenella minuta bile salt hydrolase (BSH) enzyme with at least 90% identity to SEQ ID NO: 1, wherein the modified BSH enzyme has at least one substitution modification relative to SEQ ID NO: 1.


SEQUENCE LISTING (79 sequences)

SEQ ID NO: 1 - protein, 325 aa, Christensenella minuta
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 2 - protein, 325 aa, synthetic construct
MSTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 3 - protein, 325 aa, synthetic construct
MCTAITYKTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 4 - protein, 325 aa, synthetic construct
MCTAITYYTKDHYFGRNLDLEFSYNETVTVTPKNYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 5 - protein, 325 aa, synthetic construct
MCTAITYYTKDHYFGRNLDLEFSYNETVTVTPKYYPFHFRNGKVLNHHYAMIGMAYVVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 6 - protein, 325 aa, synthetic construct
MCTAITYYTKDHYFGRNLDLEFSYNETVTVTPKYYPFHFRNGKVLNHHYAMIGMAYIVDDFPLSYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 7 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 8 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLAPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 9 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSPEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 10 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYGRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 11 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 12 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYPNY

SEQ ID NO: 13 - protein, 325 aa, synthetic construct
MCTAITYTTKDHYFGRNLDLEFSYNETVTVTPKYYPFHFRNGKVLNHHYAMIGMAAIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 14 - protein, 325 aa, synthetic construct
MCTAITYQTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNVNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 15 - protein, 325 aa, synthetic construct
MCTAITYKTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 16 - protein, 325 aa, synthetic construct
MCTAITYYTKDHYFGRNLDLEFSYNETVTVTPKYYPFHFRNGKVLNHHYAMIGMAYVVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 17 - protein, 325 aa, synthetic construct
MCTAITYYTKDHYFGRNLDLEFSYNETVTVTPKNYPFHFRNGKVLNSHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 18 - protein, 325 aa, synthetic construct
MCTAITYYTKDHYFGRNLDLEFSYNETVTVTPKNYPFHFRNGKVLKHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 19 - protein, 325 aa, synthetic construct
MCTAITYYTKDHYFGRNLDLEFSYNETVTVTPKNYPFHFRNGKVLNHHYAMIGMAYIVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 20 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYQNY

SEQ ID NO: 21 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLAPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 22 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYTNY

SEQ ID NO: 23 - protein, 325 aa, synthetic construct
MCTAITYKTKDHYFGRNLDLEFSYNETVTVTPKNYPFHFRNGKVLNHHYAMIGMAMIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 24 - protein, 325 aa, synthetic construct
MCTAITYQTKDHYFGRNLDLEFSYNETVTVTPKYYPFHFRNGKVLNHHYAMIGMATIVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 25 - protein, 325 aa, synthetic construct
MCTAITYYTKDHYFGRNLDLEFSYNETVTVTPKNYPFHFRNGKVLKHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 26 - protein, 325 aa, synthetic construct
MCTAITYYTKDHYFGRNLDLEFSYNETVTVTPKNYPFHFRNGKVLNSHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 27 - protein, 325 aa, synthetic construct
MCTAITYYTKDHYFGRNLDLEFSYNETVTVTPKNYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSPEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 28 - protein, 325 aa, synthetic construct
MCTAITYYTKDHYFGRNLDLEFSYNETVTVTPKNYPFHFRNGKVLNHHYAMIGMAYVVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 29 - protein, 325 aa, synthetic construct
MCTAITYYTKDHYFGRNLDLEFSYNETVTVTPKNYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLTPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 30 - protein, 325 aa, synthetic construct
MCTAITYYTKDHYFGRNLDLEFSYNETVTVTPKYYPFHFRNGKVLNHHYAMIGMAYVVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 31 - protein, 325 aa, synthetic construct
MCTAITYYTKDHYFGRNLDLEFSYNETVTVTPKYYPFHFRNGKVLNHHYAMIGMAYVVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYQNY

SEQ ID NO: 32 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSPEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENRQITAVDMYKENLDGNTIISYPLMKEQQINYRNY

SEQ ID NO: 33 - protein, 325 aa, synthetic construct
MCTAITYKTKDHYFGRNLDLEFSYNETVTVTPKNYPFHFRNGKVLNHHYAMIGMAYIVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 34 - protein, 325 aa, synthetic construct
MCTAITYQTKDHYFGRNLDLEFSYNETVTVTPKNYPFHFRNGKVLNHHYAMIGMAYIVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYTNY

SEQ ID NO: 35 - protein, 325 aa, synthetic construct
MCTAITYKTKDHYFGRNLDLEFSYNETVTVTPKYYPFHFRNGKVLNHHYAMIGMALIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYGQGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 36 - protein, 325 aa, synthetic construct
MCTAITYVTKDHYFGRNLDLEFSYNETVTVTPKYYPFHFRNGKVLNHHYAMIGMAYIVDDFPLYYDATNEKGLSMAGLNFADNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYGQGMGAIGLPGDLSSASRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 37 - protein, 325 aa, synthetic construct
MCTAITYYTKDHYFGRNLDLEFSYNETVTVTPKKYPFHFRNGKVLNHHYAMIGMAYVVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 38 - protein, 325 aa, synthetic construct
MCTAITYYTKDHYFGRNLDLEFSYNETVTVTPKSYPFHFRNGKVLKHHYAMIGMAYIVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 39 - protein, 325 aa, synthetic construct
MCTAITYYTKDHYFGRNLDLEFSYNETVTVTPKNYPFHFRNGKVLNNHYAMIGMAYIVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 40 - protein, 325 aa, synthetic construct
MCTAITYYTKDHYFGRNLDLEFSYNETVTVTPKNYPFHFRNGKVLNHHYAMIGMAYIVDDFPLYYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDYQMFNLNNYMHLSKEPPANTFAAELELEQYGQGMGAIGLPGDLSSASRFVKAAFTKMNSVSGDSESESISQFFHILGSVEQQRGCVHLGEDKYEITIYSSCCNMDKGIYYYTTYENHQITAVDMYKENLDGNTIISYPLMKEQQINYRNY

SEQ ID NO: 41 - protein, 325 aa, synthetic construct
MCTAITYYTKDHYFGRNLDLEFSYNETVTVTPKYYPFHFRNGKVLNHHYAMIGMATVVDDFPLYYDATNEKGLSMAGLNFPDNADYKEVKEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYRNY

SEQ ID NO: 42 - protein, 325 aa, synthetic construct
MCTAITYYTKDHYFGRNLDLEFSYNETVTVTPKYYPFHFRNGKVLNHHYAMIGMAYIVDDFPLYYDATNEKGLSMAGLNFPDNADYKEVEEGYDNIAPFEFIPWILGQCASVSEARILLEQINLVNINFSEELPLSPLHWMISDQRDSIVVESTKDGLKVFENPVGVLTNNPTFDYQMFNLNNYMHLSKEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYENY

SEQ ID NO: 43 - protein, 325 aa, synthetic construct
MCTAITYYTKDHYFGRNLDLEFSYNETVTVTPKNYPFHFRNGKVLNNHYAMIGMAYIVDDFPLFYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDKQMFNLNNYMHLSKEPPANTFAAELELEQYSRGMGAIGLPGDLSSPSRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 44 - protein, 325 aa, synthetic construct
MCTAITYYTKDHYFGRNLDLEFSYNETVTVTPKNYPFHFRNGKVLKNHYAMIGMAYIVDDFPLFYDATNEKGLSMAGLNFPDNADYKEVKEGYDNIAPFEFIPWILGQCASVSEARILLEQINLVNLNFSEELPLSPLHWMISDQRDSIVVESTKDGLKVFENPVGVLTNNPTFDKQMFNLNNYMHLSKEPPANTFAAELELEQYSRGMGAIGLPGDLSSLSRFVKAAFTKMNSVSGDSESESISQFFHILGSVEQQRGCVHLGEDKYEITIYSSCCNMDKGIYYYTTYENNQITAVDMYKENLDGNTIISYPLMKEQQINYRNY

SEQ ID NO: 45 - protein, 325 aa, synthetic construct
MCTAITYTTKDHYFGRNLDLEFSYNETVTVTPKNYPFHFRNGKVLNHHYAMIGMAVVVDDFPLYYDATNEKGLSMAGLNFPDNADYKEVKEGYDNIAPYEFIPWILGQCASVSEARILLEQINLVNINFSEELPLAPLHWMISDQRDSIVVESTKDGLKVFENPVGVLTNNPTFDYQMFNLNNYMHLSNEPPANTFAAELELEQYSRGMGAIGLPGDLSSASRFVKAAFTKMNSVSGDSESESISQFFHILGSVEQQRGCVHLGEDKYEITIYSSCCNMDKGIYYYTTYENSQITAVDMYKENLDGNTIISYPLMKEQQINYLNY

SEQ ID NO: 46 - protein, 325 aa, synthetic construct
MCTAVTYRSKHTFFGMNYDASSSEGEKIVIVPRKYPLKLSNGEVISNHYAMIGIAKVIDNYPLFKMACNEKGLAMAAISFPGIASYGEPTPGKENVAPYELIPYILGRCGSVDEALELLKTLNIVNVDFSAEHPLKPVKYMISDKEKSIVLEQTKDGLKVYENPTGVLTGNPTYDYQLFNLNNYMHLSNKPPANKFSPSIKLERISSGMGNIGLPGDYSDLSKFIRASFVRLNAVSGDSYDEQLMQFFHILDSVRNVPGTVHLGGDKYLRTLYQTCIDMDRGILYYRTYNNTQITAVDMNKCDLNGDKIIEFPLVTEPQIYELKK

SEQ ID NO: 47 - protein, 325 aa, synthetic construct
MCTAVTYKSKSSWFGMNYDASSSENEEIVIVPRKYPLKLSNGTVIDEHNAMIGIAEIRDNFPIFKMATNEKGLSMAAVSLPGIASYGEKTEGKENIAPYELIPYILGRASSVEEALKLLENLNIVNVDFSEERPLKPVRFMIADKDKAIVVEQTKDGLKVYENPTGVLTGNPLYDYQLFNLNNYMHLSNAPPANKFAPNIKLERISAGDGNIGLPGDKSDLSKFIRATFVRLNAVSGDTEDEQLMQFFHILDSVRDVPGTVWLGGDKYLRTLYQVAINMDTGKLYYRTYNNSQINMVDMNKCDLNSDKIISFPLQKELDIKELNK

SEQ ID NO: 48 - DNA, 300 bp, synthetic construct
  source 1..300: mol_type = other DNA
tgatctggaagaagcaatgaaagctgctgttaagtctccgaatcaggtattgttcctgacaggtgtattcccatccggtaaacgcggatactttgcagttgatctgactcaggaataaattataaattaaggtaagaagattgtaggataagctaatgaaatagaaaaaggatgccgtcacacaacttgtcggcattcttttttgttttattagttgaaaatatagtgaaaaagttgcctaaatatgtatgttaacaaattatttgtcgtaactttgcactccaaatctgtttttaaaga

SEQ ID NO: 49 - DNA, 200 bp, synthetic construct
  source 1..200: mol_type = other DNA
ttttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgaaataaagacatataaaagaaaagacacc

SEQ ID NO: 50 - DNA, 137 bp, synthetic construct
  source 1..137: mol_type = other DNA
aagaaaaggcgttttgtttttcttctttaccttctttccctttcgctaagagagtctgagaaacgatagaaaaagaaaagcgaaaaaacttccgaaaacatttggtagttaaaataaaacctcttacctttgcaccc

SEQ ID NO: 51 - DNA, 172 bp, synthetic construct
  source 1..172: mol_type = other DNA
ttttgcacccgctttccaagagaagaaagccttgttaaattgacttagtgtaaaagcgcagtactgcttgaccataagaacaaaaaaacttccgataaagtttggaagataaagctaaaagttcttatctttgcagtccgaaataaagacatataaaagaaaagacaccatg

SEQ ID NO: 52 - DNA, 138 bp, synthetic construct
  source 1..138: mol_type = other DNA
ttttgcacccgctttccaagagaagaaagccttgttaaattgacttagtgtaaaagcgcagtactgcttgaccataagaacaaaaaaacttccgataaagtttggaagataaagctaaaagttcttatctttgcagtc

SEQ ID NO: 53 - DNA, 30 bp, synthetic construct
  source 1..30: mol_type = other DNA
gaaataaagacatataaaagaaaagacacc

SEQ ID NO: 54 - DNA, 54 bp, synthetic construct
  source 1..54: mol_type = other DNA
cgcattttaaaataaaataaattatttatgatattaaacgaatgaaaagacacc

SEQ ID NO: 55 - DNA, 54 bp, synthetic construct
  source 1..54: mol_type = other DNA
cgcattttaaaataaaataaataatttacttaattaaacgaatgaaaagacacc

SEQ ID NO: 56 - DNA, 54 bp, synthetic construct
  source 1..54: mol_type = other DNA
cgcattttaaaataaaataaattatttatttaattaaacgaatgaaaagacacc

SEQ ID NO: 57 - DNA, 81 bp, synthetic construct
  source 1..81: mol_type = other DNA
atgagatttattaaacaagtacttttgactataacattatgtaacataatgttatttgcattgccatctactgtaaatgca

SEQ ID NO: 58 - DNA, 4318 bp, synthetic construct
  source 1..4318: mol_type = other DNA
ggcggaagatgtccattattttatggacgaaggagtcgatctgctgattatttccgctaacgaagctgctcctatgactccgattgtagaagaagcttatcagaaaggcatccccgttattcttgtagaccggaagattctttcggataaatatactgcctatatcggagccgataattatgaaatcggtcgttcggtaggaaactatattgcctccagtctgaaagggaagggaaatatagtagaactgacaggattgagcggttcgactcctgcaatggaacggcatcagggatttatggctgccatcagtaaattcccggatataaagctgattgataaagcggatgctgcgtgggaacgtggtccggcagagatagaaatggatagtatgcttcggaggcatcctaagattgatgctgtgtatgcccataatgaccgtatcgctccgggtgcctatcaggcagcaaagatggcagggcgggagaaggaaatgatttttgtcggcatagatgccttgccgggtaagggaaacggactggaactggttttggacagtgtgctggatgccacctttatctatccgaccaatggcgataaggtactgcaactggctatggacattctggagaagaaaccctatcccaaagaaacggtgatgaataccgctgttgtggaccgtaccaacgcacacgtcatgcagttgcagactacacacatctccgaactcgataaaaagattgaaacgctcaacggacgtatcggtggatacctctctcaggtagctacacaacaggtcgttttatacggcagtctgattatccttttattggtagccggcttattattggtcgtttataaatcactccgctctaagaatcgcttgaataaagagctttttaagcagaagcagcaattggaagagcagcgtgacaaactggaagaacagcgtgaccaattgatacagctctctcatcaactggaagaagctacccatgccaagctggtcacgcgtaaacatttacagttgcatgtggcctattgtttttagccgttaaatattttataactattaaatagcgatacaaattgttcgaaactaatattgtttatatcatatattctcgcatgttttaaagctttattaaattgattttttgtaaacagtttttcgtactctttgttaacccatttcattacaaaagtttcatatttttttctctctttaaatgccatttttgctggctttctttttaatacaattaatgtgctatccactttaggttttggatggaaataatacctaggaatttttgctaatatagaaatatctacctctgccattaacagcaatgctagtgatctgtttgtatctaataacattttagcaaaaccatattccactattaaataacttattgtggctgaactttcaaaaacaatttttcgaattatatttgtgcttatgttgtaaggtatgctgccaaatattttatatggattgtggctaggaaatgtaaatttcagtatatcatcatttactatttgatagttaggataatttaagagcttattacgagttacctcacataatttagaatcaatttctatcgccgttacaaaattacatctctttaccaatccagcagtaaaatgacctttccctgcacctatttcaaagatgttatctttttcatctaaacttatgcaattcattattttttctatgtgatattttgaagtaataaaattttgactatcttttatatttactttgttcattataacctctccttaatttattgcatctcttttcgaatatttatgttttttgagaaaagaacgtactcatggttcatcccgatatgcgtatcggtctgtatatcagcaactttctatgtgtttcaactacaatagtcatctattctcatctttctgagtccaccccctgcaaagcccctctttacgacataaaaattcggtcggaaaaggtatgcaaaagatgtttctctctttaagagaaactcttcgggatgcaaaaatatgaaaataactccaattcaccaaattatatagcgacttttttacaaaatgctaaaatttgttgatttccgtcaagcaattgttgagcaaaaatgtcttttacgataaaatgatacctcaatatcaactgtttagcaaaacgatatttctcttaaagagagaaacacctttttgttcaccaatccccgacttttaatcccgcggccatgattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcataacgcgtcaattcgagggggatcaattccgtgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaacggaattgatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattcccctccaccgcggtggcggccgctctagaactagtggatcccccgggctgcaggaattcgatatcaagcttatcgataccgtcgacgtcgactaattgcctatcttccagtgatggaacagcatttgtgcattggctgcaacaatcagccttacttgtgcctgttctatttccgaaccgaccgcttgtatgaatccatcaaaattcgttttctctatgttggattccttgttgctcatattgtgatgataatttctacaaatatagtcattggtaactatctatgaaactgtttgatacctagcataaccccttggggcctctaaacgggtcttgaggagaaggccatcctgacggatggccttttggttttttgtttta

SEQ ID NO: 59 - DNA, 300 bp, synthetic construct; identical to SEQ ID NO: 48
  source 1..300: mol_type = other DNA

SEQ ID NO: 60 - DNA, 200 bp, synthetic construct; identical to SEQ ID NO: 49
  source 1..200: mol_type = other DNA

SEQ ID NO: 61 - DNA, 191 bp, synthetic construct
  source 1..191: mol_type = other DNA
aagaaaaggcgttttgtttttcttctttaccttctttccctttcgctaagagagtctgagaaacgatagaaaaagaaaagcgaaaaaacttccgaaaacatttggtagttaaaataaaacctcttacctttgcaccccgcattttaaaataaaataaattatttatgatattaaacgaatgaaaagacacc

SEQ ID NO: 62 - DNA, 5215 bp, synthetic construct
  source 1..5215: mol_type = other DNA
ggaattcgatatcaagcttatcgataccgtcgacgtcgactaattgcctatcttccagtgatggaacagcatttgtgcattggctgcaacaatcagccttacttgtgcctgttctatttccgaaccgaccgcttgtatgaatccatcaaaattcgttttctctatgttggattccttgttgctcatattgtgatgataatttctacaaatatagtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtccttcttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcgctggtgtctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgctctttacccgctgcttatctgcatcccatttggctacgtcaatccggtagcccgttgtaaactcgatgcgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcggctgtttttgttttatttaagataatacaaccactacataataaaagagtagcgatattaaaagaatccgatgagaaaagactaatatttatctatccattcagtttgatttttcaggactttacatcgtcctgaaagtatttgttggtaccggtaccgaggacgcgtaaacatttacagttgcatgtggcctattgtttttagccgttaaatattttataactattaaatagcgatacaaattgttcgaaactaatattgtttatatcatatattctcgcatgttttaaagctttattaaattgattttttgtaaacagtttttcgtactctttgttaacccatttcattacaaaagtttcatatttttttctctctttaaatgccatttttgctggctttctttttaatacaattaatgtgctatccactttaggttttggatggaaataatacctaggaatttttgctaatatagaaatatctacctctgccattaacagcaatgctagtgatctgtttgtatctaataacattttagcaaaaccatattccactattaaataacttattgtggctgaactttcaaaaacaatttttcgaattatatttgtgcttatgttgtaaggtatgctgccaaatattttatatggattgtggctaggaaatgtaaatttcagtatatcatcatttactatttgatagttaggataatttaagagcttattacgagttacctcacataatttagaatcaatttctatcgccgttacaaaattacatctctttaccaatccagcagtaaaatgacctttccctgcacctatttcaaagatgttatctttttcatctaaacttatgcaattcattattttttctatgtgatattttgaagtaataaaattttgactatcttttatatttactttgttcattataacctctccttaatttattgcatctcttttcgaatatttatgttttttgagaaaagaacgtactcatggttcatcccgatatgcgtatcggtctgtatatcagcaactttctatgtgtttcaactacaatagtcatctattctcatctttctgagtccaccccctgcaaagcccctctttacgacataaaaattcggtcggaaaaggtatgcaaaagatgtttctctctttaagagaaactcttcgggatgcaaaaatatgaaaataactccaattcaccaaattatatagcgacttttttacaaaatgctaaaatttgttgatttccgtcaagcaattgttgagcaaaaatgtcttttacgataaaatgatacctcaatatcaactgtttagcaaaacgatatttctcttaaagagagaaacacctttttgttcaccaatccccgacttttaatcccgcggccatgattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcataacgcgtcaattcgagggggatcaattccgtgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaacggaattgatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattcccctccaccgcggtggcggccgctctagaactagtggatcccccgggctgcatgatctggaagaagcaatgaaagctgctgttaagtctccgaatcaggtattgttcctgacaggtgtattcccatccggtaaacgcggatactttgcagttgatctgactcaggaataaattataaattaaggtaagaagattgtaggataagctaatgaaatagaaaaaggatgccgtcacacaacttgtcggcattcttttttgttttattagttgaaaatatagtgaaaaagttgcctaaatatgtatgttaacaaattatttgtcgtaactttgcactccaaatctgtttttaaaga

SEQ ID NO: 63 - DNA, 5115 bp, synthetic construct
  source 1..5115: mol_type = other DNA
ggaattcgatatcaagcttatcgataccgtcgacgtcgactaattgcctatcttccagtgatggaacagcatttgtgcattggctgcaacaatcagccttacttgtgcctgttctatttccgaaccgaccgcttgtatgaatccatcaaaattcgttttctctatgttggattccttgttgctcatattgtgatgataatttctacaaatatagtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtccttcttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcgctggtgtctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgctctttacccgctgcttatctgcatcccatttggctacgtcaatccggtagcccgttgtaaactcgatgcgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcggctgtttttgttttatttaagataatacaaccactacataataaaagagtagcgatattaaaagaatccgatgagaaaagactaatatttatctatccattcagtttgatttttcaggactttacatcgtcctgaaagtatttgttggtaccggtaccgaggacgcgtaaacatttacagttgcatgtggcctattgtttttagccgttaaatattttataactattaaatagcgatacaaattgttcgaaactaatattgtttatatcatatattctcgcatgttttaaagctttattaaattgattttttgtaaacagtttttcgtactctttgttaacccatttcattacaaaagtttcatatttttttctctctttaaatgccatttttgctggctttctttttaatacaattaatgtgctatccactttaggttttggatggaaataatacctaggaatttttgctaatatagaaatatctacctctgccattaacagcaatgctagtgatctgtttgtatctaataacattttagcaaaaccatattccactattaaataacttattgtggctgaactttcaaaaacaatttttcgaattatatttgtgcttatgttgtaaggtatgctgccaaatattttatatggattgtggctaggaaatgtaaatttcagtatatcatcatttactatttgatagttaggataatttaagagcttattacgagttacctcacataatttagaatcaatttctatcgccgttacaaaattacatctctttaccaatccagcagtaaaatgacctttccctgcacctatttcaaagatgttatctttttcatctaaacttatgcaattcattattttttctatgtgatattttgaagtaataaaattttgactatcttttatatttactttgttcattataacctctccttaatttattgcatctcttttcgaatatttatgttttttgagaaaagaacgtactcatggttcatcccgatatgcgtatcggtctgtatatcagcaactttctatgtgtttcaactacaatagtcatctattctcatctttctgagtccaccccctgcaaagcccctctttacgacataaaaattcggtcggaaaaggtatgcaaaagatgtttctctctttaagagaaactcttcgggatgcaaaaatatgaaaataactccaattcaccaaattatatagcgacttttttacaaaatgctaaaatttgttgatttccgtcaagcaattgttgagcaaaaatgtcttttacgataaaatgatacctcaatatcaactgtttagcaaaacgatatttctcttaaagagagaaacacctttttgttcaccaatccccgacttttaatcccgcggccatgattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcataacgcgtcaattcgagggggatcaattccgtgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaacggaattgatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattcccctccaccgcggtggcggccgctctagaactagtggatcccccgggctgcacgaatataagccggatatcgaaattaaattgctctatcatgaaattattgtttaaacaactttgcaaaggtaagccacttcccggataattcctacattaggatacaaaaaaagattaaatatttgtagttcaaaataaaatgcctaactttgcacccgcattttaaaataaaataaattatttatttaattaaacgagt

SEQ ID NO: 64 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaatactatccgtttcattttcgtaatggcaaggtgctgaatcatcattatgcaatgattggcatggcctacatcgttgatgattttccgctgtattatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacctgaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaaagaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacaatcagattaccgcagtggatatgtacaaagaaaatctggatggcaacaccattattagctacccgctgatgaaagaacagcagattaactatcgcaattattaa

SEQ ID NO: 65 - DNA, 978 bp, synthetic construct
  source 1..978: mol_type = other DNA
atgagcaccgccattacctattataccaaagatcattatttcggtcgcaacctggatctggaattttcctataatgaaaccgttaccgtgacgccgaaatactatccgtttcattttcgtaatggcaaggtgctgaatcatcattatgcaatgattggcatggcctacatcgttgatgattttccgctgtattatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacctgaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaaagaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacaatcagattaccgcagtggatatgtacaaagaaaatctggatggcaacaccattattagctacccgctgatgaaagaacagcagattaactatcgcaattattaa

SEQ ID NO: 66 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaatactatccgtttcattttcgtaatggcaaggtgctgaatcatcattatgcaatgattggcatggcctacgttgttgatgattttccgctgtattatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacctgaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaaagaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacaatcagattaccgcagtggatatgtacaaagaaaatctggatggcaacaccattattagctacccgctgatgaaagaacagcagattaactatcgcaattattaa

SEQ ID NO: 67 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaatactatccgtttcattttcgtaatggcaaggtgctgaatcatcattatgcaatgattggcatggcctacatcgttgatgattttccgctgagctatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacctgaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaaagaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacaatcagattaccgcagtggatatgtacaaagaaaatctggatggcaacaccattattagctacccgctgatgaaagaacagcagattaactatcgcaattattaa

SEQ ID NO: 68 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaatactatccgtttcattttcgtaatggcaaggtgctgaatcatcattatgcaatgattggcatggcctacatcgttgatgattttccgctgtattatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacctgaactttagcgaagaactgccgctggcaccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaaagaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacaatcagattaccgcagtggatatgtacaaagaaaatctggatggcaacaccattattagctacccgctgatgaaagaacagcagattaactatcgcaattattaa

SEQ ID NO: 69 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaatactatccgtttcattttcgtaatggcaaggtgctgaatcatcattatgcaatgattggcatggcctacatcgttgatgattttccgctgtattatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacctgaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcccggaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacaatcagattaccgcagtggatatgtacaaagaaaatctggatggcaacaccattattagctacccgctgatgaaagaacagcagattaactatcgcaattattaa

SEQ ID NO: 70 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaatactatccgtttcattttcgtaatggcaaggtgctgaatcatcattatgcaatgattggcatggcctacatcgttgatgattttccgctgtattatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacattaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcccggaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaaccgtcagattaccgcagtggatatgtacaaagaaaatctggatggcaacaccattattagctacccgctgatgaaagaacagcagattaactatcgcaattattaa

SEQ ID NO: 71 - DNA, 978 bp, synthetic construct
  source 1..978: mol_type = other DNA
atgtgtaccgccattacctatgttaccaaagatcattatttcggtcgcaacctggatctggaattttcctataatgaaaccgttaccgtgacgccgaaatactatccgtttcattttcgtaatggcaaggtgctgaatcatcattatgcaatgattggcatggcctacatcgttgatgattttccgctgtattatgatgccaccaatgaaaaaggtctgagcatggcaggtctgaattttgcagataatgccgattataaagaagtgaaagaaggctacgataacattgcaccgtttgagtttattccgtggattctgggtcagtgtgcaagcgttagcgaagcacgtattctgctggaacaaattaatctggtgaacctgaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaaagaaccgcctgcaaatacctttgcagcagaactggaattagaacagtatggtcagggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacaatcagattaccgcagtggatatgtacaaagaaaatctggatggcaacaccattattagctacccgctgatgaaagaacagcagattaactatcgcaattattaa

SEQ ID NO: 72 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaaaaatatccgtttcattttcgtaatggcaaggtgctgaatcatcattatgcaatgattggcatggcctacgttgttgatgattttccgctgtattatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacattaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaaagaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacagccagattaccgcagtggatatgtacaaagaaaatctggatggcaacaccattattagctacccgctgatgaaagaacagcagattaactatcgcaattattaa

SEQ ID NO: 73 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaaagctatccgtttcattttcgtaatggcaaggtgctgaaacatcattatgcaatgattggcatggcctacatcgttgatgattttccgctgtattatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacattaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaaagaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacagccagattaccgcagtggatatgtacaaagaaaatctggatggcaacaccattattagctacccgctgatgaaagaacagcagattaactatcgcaattattaa

SEQ ID NO: 74 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaatactatccgtttcattttcgtaatggcaaggtgctgaatcatcattatgcaatgattggcatggccaccgttgttgatgattttccgctgtattatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacattaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaaagaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacagccagattaccgcagtggatatgtacaaagaaaatctggatggcaacaccattattagctacccgctgatgaaagaacagcagattaactatcgcaattattaa

SEQ ID NO: 75 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaatactatccgtttcattttcgtaatggcaaggtgctgaatcatcattatgcaatgattggcatggcctacatcgttgatgattttccgctgtattatgatgccaccaatgaaaaaggtctgagcatggcaggtctgaattttccggataatgccgattataaagaagtggaagaaggctacgataacattgcaccgtttgagtttattccgtggattctgggtcagtgtgcaagcgttagcgaagcacgtattctgctggaacaaattaatctggtgaacattaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaaagaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacagccagattaccgcagtggatatgtacaaagaaaatctggatggcaacaccattattagctacccgctgatgaaagaacagcagattaactatgaaaattattaa

SEQ ID NO: 76 - DNA, 978 bp, synthetic construct
  source 1..978: mol_type = other DNA
atgtgtaccgccattacctattataccaaagatcattatttcggtcgcaacctggatctggaattttcctataatgaaaccgttaccgtgacgccgaaaaattatccgtttcattttcgtaatggcaaggtgctgaaaaatcattatgcaatgattggcatggcctacatcgttgatgattttccgctgttttatgatgccaccaatgaaaaaggtctgagcatggcaggtctgaattttccggataatgccgattataaagaagtgaaagaaggctacgataacattgcaccgtttgagtttattccgtggattctgggtcagtgtgcaagcgttagcgaagcacgtattctgctggaacaaattaatctggtgaacctgaactttagcgaagaactgccgctgagtccgctgcattggatgattagcgatcagcgtgatagcattgttgttgaaagcaccaaagatggcctgaaagtttttgaaaatccggttggtgttctgaccaataatccgacctttgataaacagatgttcaacctgaacaactacatgcacctgagcaaagaaccgcctgcaaatacctttgcagcagaactggaattagaacagtatagccgtggtatgggtgcaattggtctgcctggtgatctgagcagcctgagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacaatcagattaccgcagtggatatgtacaaagaaaatctggatggcaacaccattattagctacccgctgatgaaagaacagcagattaactatcgcaattattaa

SEQ ID NO: 77 - DNA, 978 bp, synthetic construct
  source 1..978: mol_type = other DNA
atgtgtaccgccattacctataccaccaaagatcattatttcggtcgcaacctggatctggaattttcctataatgaaaccgttaccgtgacgccgaaaaattatccgtttcattttcgtaatggcaaggtgctgaatcatcattatgcaatgattggcatggccgttgttgttgatgattttccgctgtattatgatgccaccaatgaaaaaggtctgagcatggcaggtctgaattttccggataatgccgattataaagaagtgaaagaaggctacgataacattgcaccgtatgagtttattccgtggattctgggtcagtgtgcaagcgttagcgaagcacgtattctgctggaacaaattaatctggtgaacattaactttagcgaagaactgccgctggcaccgctgcattggatgattagcgatcagcgtgatagcattgttgttgaaagcaccaaagatggcctgaaagtttttgaaaatccggttggtgttctgaccaataatccgacctttgattatcagatgttcaacctgaacaactacatgcacctgagcaatgaaccgcctgcaaatacctttgcagcagaactggaattagaacagtatagccgtggtatgggtgcaattggtctgcctggtgatctgagcagcgcaagccgttttgttaaagcagcatttaccaaaatgaatagcgtgagcggtgatagcgaaagcgaaagcattagccagtttttccatattctgggtagcgttgaacagcagcgtggttgtgttcatctgggtgaagataaatatgagatcaccatttattcgagctgctgcaatatggataaaggcatctattattacaccacctacgagaacagccagattaccgcagtggatatgtacaaagaaaatctggatggcaacaccattattagctacccgctgatgaaagaacagcagattaactatctgaattattaa

SEQ ID NO: 78 - DNA, 978 bp, synthetic construct
  source 1..978: mol_type = other DNA
atgtgtaccgccgttacctatcgtagcaaacatacctttttcggtatgaactatgatgcaagcagctccgaaggtgaaaaaattgttattgttccgcgtaaatatccgctgaaactgagcaatggcgaagtgattagcaatcattatgcaatgattggcattgccaaagttattgataattatccgctgtttaaaatggcctgtaatgaaaaaggtctggcaatggcagcaattagctttccgggtattgccagctatggtgaaccgaccccgggcaaagaaaacgttgcaccgtatgagctgattccgtatattctgggtcgttgtggtagcgttgatgaagcactggaactgctgaaaaccctgaatattgtgaacgttgattttagcgcagaacatccgctgaaaccggttaaatatatgattagcgataaagaaaaaagcattgttctggaacagaccaaagatggcctgaaagtttatgaaaatccgaccggtgttctgaccggtaatccgacctatgattatcagctgttcaacctgaacaactacatgcacctgagcaataaaccgcctgcaaataaatttagcccgagcattaaattagaacgtattagcagcggtatgggtaatattggtctgcctggtgattatagcgatctgagcaaatttattcgtgcaagctttgttcgtctgaatgcagtgagcggtgatagctatgatgaacagctgatgcagtttttccatattctggatagcgttcgtaatgttccgggtaccgttcatctgggtggtgataaatatctgcgtaccctgtatcagacctgcattgatatggatcgtggcatcctgtattaccgtacctacaataacacccagattaccgcagtggatatgaataaatgtgatctgaatggcgataaaattattgaatttccgctggttaccgaaccgcagatttatgaactgaaaaaataa

SEQ ID NO: 79 - DNA, 978 bp, synthetic construct
  source 1..978: mol_type = other DNA
atgtgtaccgccgttacctataaaagcaaaagcagctggttcggtatgaactatgatgcaagcagctccgaaaatgaagaaattgttattgttccgcgtaaatatccgctgaaactgagcaatggcaccgtgattgatgaacataatgcaatgattggcattgccgaaatccgtgataattttccgatttttaaaatggccaccaatgaaaaaggtctgagcatggcagcagttagcctgccgggtattgccagctatggtgaaaaaaccgaaggcaaagaaaacattgcaccgtatgagctgattccgtatattctgggtcgtgcaagcagcgttgaagaagcactgaaactgctggaaaatctgaatattgtgaacgttgattttagcgaagaacgtccgctgaaaccggttcgttttatgattgcagataaagataaagcaattgttgttgaacagaccaaagatggcctgaaagtttatgaaaatccgaccggtgttctgaccggtaatccgctgtatgattatcagctgttcaacctgaacaactacatgcacctgagcaatgcaccgcctgcaaataaatttgcaccgaatattaaattagaacgtattagcgcaggtgatggtaatattggtctgcctggtgataaaagcgatctgagcaaatttattcgtgcaacctttgttcgtctgaatgcagtgagcggtgataccgaagatgaacagctgatgcagtttttccatattctggatagcgttcgtgatgttccgggtaccgtttggctgggtggtgataaatatctgcgtaccctgtatcaggttgcaattaatatggataccggcaaactgtattaccgtacctacaataacagccagattaatatggtggatatgaataaatgtgatctgaatagcgataaaattattagctttccgctgcagaaagaactggatattaaagaactgaataaataa